(19) United States
(12) Patent Application Publication
(10) Pub. No.: US 2023/0012167 A1
(43) Pub. Date: Jan. 12, 2023

(54) INHIBITORS OF BRUTON'S TYROSINE KINASE AND METHODS OF THEIR USE

(71) Applicant: Janssen Pharmaceutica NV, Beerse (BE)

(72) Inventors: Jacqueline Cirillo BUSSOLARI, Skillman, NJ (US); Ulrike PHILIPPAR, Antwerpen (BE); Andrew STEELE, Doylestown, PA (US); Mark S. TICHENOR, San Diego, CA (US)

(21) Appl. No.: 17/854,801

(22) Filed: Jun. 30, 2022

Related U.S. Application Data
(60) Provisional application No. 63/216,796, filed on Jun. 30, 2021.

(51) Int. Cl. A61K 31/522; A61K 31/501; A61K 45/06

(52) U.S. Cl. CPC A61K 31/522; A61K 31/501; A61K 45/06

(57) ABSTRACT

The present disclosure is directed to the use of a compound of Formula (III)

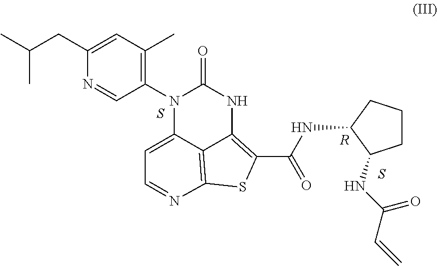

in the treatment of DLBCL.

### CLAIM OF PRIORITY

This application claims priority to U.S. Provisional Application No. 63/216,796 filed on Jun. 30, 2021 titled “Inhibitors Of Bruton's Tyrosine Kinase And Methods Of Their Use” which is incorporated hereby by reference in its entiriety.

### TECHNICAL FIELD

The present disclosure is directed to the use of small molecule tyrosine kinase inhibitors for the treatment of diffuse large B-cell lymphoma (DLBCL).

### BACKGROUND

Malignancies, in particular DLBCL continue to afflict patients. DLBCL is the most prevalent type of aggressive non-Hodgkin's lymphoma (NHL) in the United States. Activated B-cell diffuse large B-cell lymphoma (ABC-DLBCL) accounts for approximately 30% total DLBCL diagnoses. While the majority of the patients with DLBCL show response to the initial treatment, approximately one-third of patients have refractory disease or experience relapse after the standard therapies. B cell receptor (BCR) signaling is an important growth and survival pathway in various B cell malignancies, including DLBCL. Alternative, effective treatments of cancer are still needed. Human Bruton's tyrosine kinase (“BTK”) is a ˜76 kDa protein belonging to the Tec family of non-receptor tyrosine kinases. Tec kinases form the second largest family of cytoplasmic tyrosine kinases in mammalian cells, which consists of four other members in addition to BTK: the eponymous kinase TEC, ITK, TXK/RLK and BMX. Tec kinases are evolutionarily conserved throughout vertebrates. They are related to, but structurally distinct from, the larger Src and Syk kinase families. Tec family proteins are abundantly expressed in hematopoietic tissues and play important roles in the growth and differentiation of blood and endothelial cells in mammals. Based upon BTK expression from IHC studies described in the art, Btk inhibition has the potential to modulate biology associated with B cells, macrophages, mast cells, osteoclasts, and platelet microparticles. Cometh, O. B., et al. Curr. Top. Microbiol. Immunol. BTK Signaling in B Cell Differentiation and Autoimmunity. 2015 Sep. 5.

### SUMMARY

Disclosed herein are methods of treating DLBCL in a subject comprising (a) determining an expression level or a modification of one or more biomarker genes selected from CCL3, CCL4, ACTG2, LOR, GAPT, CCND2, SELL, GEN1, HDAC9, CD10, BCL6, MUM1, MYD88, CD79B, PIM1, CDKN2A, HLA-B, OSBPL10, ETV6, SPIB, TOX, BTG1, BTG2, HLA-A, SETD1B, HLA-C, MPEG1, FOXC1, TBL1XR1, KLHL14, GRHPR, CD58, PRDM1, VMP1, PIM2, WEE1, BCL11A, CHST2, ARID5B, HASPIN, IL16, PPP1R9B, HNF1B, or any combination thereof, in a sample from the patient; and (b) administering a therapeutically effective amount compound of Formula (III):

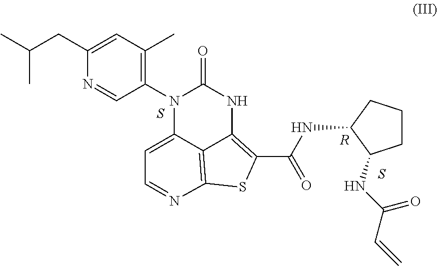

if the expression of one or more biomarker genes is increased relative to a control or reference level, or if there is a presence of modification in one or more biomarker genes. In some aspects, the control or reference level is the level of expression of the biomarker genes in a normal patient. In some embodiments, the modification is a base substitution, an insertion, a deletion, a DNA rearrangement, a translocation, a copy number alteration, or a combination thereof.

Also disclosed are methods of treating activated B-cell diffuse large B-cell lymphoma (ABC-DLBCL) in a subject comprises (a) determining an expression level or a modification of one or more biomarker genes selected from CCL3, CCL4, ACTG2, LOR, GAPT, CCND2, SELL, GEN1, HDAC9, CD10, BCL6, MUM1, MYD88, CD79B, PIM1, CDKN2A, HLA-B, OSBPL10, ETV6, SPIB, TOX, BTG1, BTG2, HLA-A, SETD1B, HLA-C, MPEG1, FOXC1, TBL1XR1, KLHL14, GRHPR, CD58, PRDM1, VMP1, PIM2, WEE1, BCL11A, CHST2, ARID5B, HASPIN, IL16, PPP1R9B, HNF1B, or any combination thereof, in a sample from the patient; and (b) administering a therapeutically effective amount of compound of Formula (III):

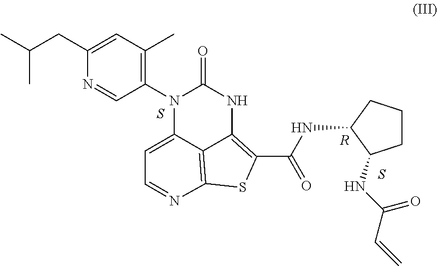

if the expression of one or more biomarker genes is increased relative to a control or reference level, or if there is a presence of modification in one or more biomarker genes. In some aspects, the control or reference level is the level of expression of the biomarker genes in a normal patient. In some embodiments, the modification is a base substitution, an insertion, a deletion, a DNA rearrangement, a translocation, a copy number alteration, or a combination thereof.

Also disclosed are methods of treating non-germinal center B-cell diffuse large B-cell lymphoma (non-GCB-DLBCL) in a subject comprises (a) determining an expression level or a modification of one or more biomarker genes selected from CCL3, CCL4, ACTG2, LOR, GAPT, CCND2, SELL, GEN1, HDAC9, CD10, BCL6, MUM1, MYD88, CD79B, PIM1, CDKN2A, HLA-B, OSBPL10, ETV6, SPIB, TOX, BTG1, BTG2, HLA-A, SETD1B, HLA-C, MPEG1, FOXC1, TBL1XR1, KLHL14, GRHPR, CD58, PRDM1, VMP1, PIM2, WEE1, BCL11A, CHST2, ARID5B, HASPIN, IL16, PPP1R9B, HNF1B, or any combination thereof, in a sample from the patient; and (b) administering a therapeutically effective amount of compound of Formula (III):

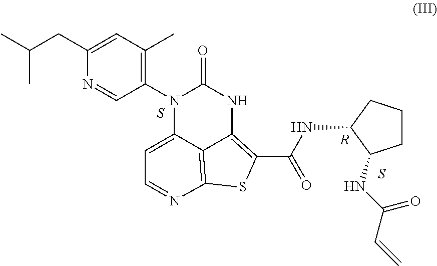

if the expression of one or more biomarker genes is increased relative to a control or reference level, or if there is a presence of modification in one or more biomarker genes. In some aspects, the control or reference level is the level of expression of the biomarker genes in a normal patient. In some embodiments, the modification is a base substitution, an insertion, a deletion, a DNA rearrangement, a translocation, a copy number alteration, or a combination thereof.

Also disclosed are methods of treating germinal center B-cell diffuse large B-cell lymphoma (GCB-DLBCL) in a subject comprises (a) determining an expression level or a modification of one or more biomarker genes selected from CCL3, CCL4, ACTG2, LOR, GAPT, CCND2, SELL, GEN1, HDAC9, CD10, BCL6, MUM1, MYD88, CD79B, PIM1, CDKN2A, HLA-B, OSBPL10, ETV6, SPIB, TOX, BTG1, BTG2, HLA-A, SETD1B, HLA-C, MPEG1, FOXC1, TBL1XR1, KLHL14, GRHPR, CD58, PRDM1, VMP1, PIM2, WEE1, BCL11A, CHST2, ARID5B, HASPIN, IL16, PPP1R9B, HNF1B, or any combination thereof, in a sample from the patient; and (b) administering a therapeutically effective amount of compound of Formula (III):

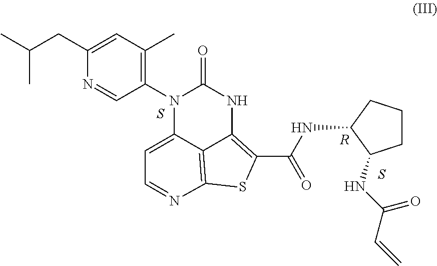

if the expression of one or more biomarker genes is increased relative to a control or reference level, or if there is a presence of modification in one or more biomarker genes. In some aspects, the control or reference level is the level of expression of the biomarker genes in a normal patient. In some embodiments, the modification is a base substitution, an insertion, a deletion, a DNA rearrangement, a translocation, a copy number alteration, or a combination thereof.

In some aspects, the therapeutically effective amount of the compound of Formula (III) is from about 140 mg to about 560 mg. In some aspects, the therapeutically effective amount of the compound of Formula (III)is about 140 mg. In some aspects, the therapeutically effective amount of the compound of Formula (III) is about 280 mg. In some aspects, the therapeutically effective amount of the compound of Formula (III) is about 560 mg. In some aspects, the therapeutically effective amount of the compound of Formula (III) is administered once a day. In some aspects, the therapeutically effective amount of the compound of Formula (III) is administered twice a day. In some aspects, the therapeutically effective amount of the compound of Formula (III) is administered three times a day. In some aspects, the compound of formula (III) is administered orally.

Some aspects further comprise administering 4-(4-{[2-(4-chlorophenyl)-4,4-dimethylcyclohex-1-en-1-yl]methyl}piperazin-1-yl)-N-({3-nitro-4-[(tetrahydro-2H-pyran-4-ylmethyl)amino]phenyl}sulfonyl)-2-(1H-pyrrolo[2,3-b]pyridin5-yloxy)benzamide). Some aspects further comprise administering cyclophosphamide, doxorubicin, vincristine, prednisone and rituximab.

### DETAILED DESCRIPTION

The disclosure may be more fully appreciated by reference to the following description, including the following glossary of terms and the concluding examples. It is to be appreciated that certain features of the disclosed compositions and methods which are, for clarity, described herein in the context of separate aspects, may also be provided in combination in a single aspect. Conversely, various features of the disclosed compositions and methods that are, for brevity, described in the context of a single aspect, may also be provided separately or in any subcombination. It will be understood by those within the art that, in general, terms used herein, and especially in the appended claims (for example, bodies of the appended claims) are generally intended as “open” terms (for example, the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to aspects containing only one such recitation, even when the same claim includes the introductory phrases “one or more” or “at least one” and indefinite articles such as “a” or “an” (for example, “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (for example, “ a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (for example, “ a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed aspect.

The term “about” as used herein when immediately preceding a numerical value means a range of plus or minus 10% of that value, for example, “about 50” means 45 to 55, “about 25,000” means 22,500 to 27,500, etc., unless the context of the disclosure indicates otherwise, or is inconsistent with such an interpretation.

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salt” refers to a salt of a compound of the disclosure that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate and the like.

“Pharmaceutically acceptable vehicle” refers to a diluent, adjuvant, excipient or carrier with which a compound of the disclosure is administered. A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of an agent and that is compatible therewith. Examples of excipients include calcium carbonate, calcium phosphate, various sugars and types of starch, cellulose derivatives, gelatin, vegetable oils, and polyethylene glycols.

“Subject” includes humans. The terms “human,” “patient,” and “subject” are used interchangeably herein.

“Treating” or “treatment” of any disease or disorder refers, in one aspect, to ameliorating the disease or disorder (i.e., arresting or reducing the development of the disease or at least one of the clinical symptoms thereof). In another aspect “treating” or “treatment” refers to ameliorating at least one physical parameter, which may not be discernible by the subject. In yet another aspect, “treating” or “treatment” refers to modulating the disease or disorder, either physically, (e.g., stabilization of a discernible symptom), physiologically, (e.g., stabilization of a physical parameter), or both. In yet another aspect, “treating” or “treatment” refers to delaying the onset of the disease or disorder.

“Compounds of the present disclosure,” and equivalent expressions, are meant to embrace compounds of the Formula (III) as described herein, which expression includes the pharmaceutically acceptable salts, and the solvates, e.g., hydrates, and polymorphs thereof, where the context so permits. Similarly, reference to intermediates, whether or not they themselves are claimed, is meant to embrace their salts, and solvates, where the context so permits.

As used herein, the term “isotopic variant” refers to a compound that contains unnatural proportions of isotopes at one or more of the atoms that constitute such compound. For example, an “isotopic variant” of a compound can be radiolabeled, that is, contain one or more non-radioactive isotopes, such as for example, deuterium (²H or D), carbon-13 (¹³C), nitrogen-15 (¹⁵N), or the like. It will be understood that, in a compound where such isotopic substitution is made, the following atoms, where present, may vary, so that for example, any hydrogen may be ²H/D, any carbon may be ¹³C, or any nitrogen may be ¹⁵N, and that the presence and placement of such atoms may be determined within the skill of the art. Likewise, the disclosure may include the preparation of isotopic variants with radioisotopes, in the instance for example, where the resulting compounds may be used for drug and/or substrate tissue distribution studies. Radiolabeled compounds of the disclosure can be used in diagnostic methods such as Single-photon emission computed tomography (SPECT). The radioactive isotopes tritium, i.e. ³H, and carbon-14, i.e. ¹⁴C, are particularly useful for their ease of incorporation and ready means of detection. Further, compounds may be prepared that are substituted with positron emitting isotopes, such as ¹¹C, ¹⁸F, ¹⁵O and ¹³N, and would be useful in Positron Emission Topography (PET) studies for examining substrate receptor occupancy.

All isotopic variants of the compounds of the disclosure, radioactive or not, are intended to be encompassed within the scope of the disclosure.

It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers,” for example, diastereomers, enantiomers, and atropisomers.

Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R-and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture.”

“Atropisomers” refer to stereoisomers that arise because of hindered rotation around a single bond.

“Tautomers” refer to compounds that are interchangeable forms of a particular compound structure, and that vary in the displacement of hydrogen atoms and electrons. Thus, two structures may be in equilibrium through the movement of it electrons and an atom (usually H). For example, enols and ketones are tautomers because they are rapidly interconverted by treatment with either acid or base. Another example of tautomerism is the aci-and nitro-forms of phenyl nitromethane, that are likewise formed by treatment with acid or base.

Tautomeric forms may be relevant to the attainment of the optimal chemical reactivity and biological activity of a compound of interest.

The compounds of this disclosure may possess one or more asymmetric centers; such compounds can therefore be produced as individual (R)-or (S)-stereoisomers or as mixtures thereof.

Unless indicated otherwise, the description or naming of a particular compound in the specification and claims is intended to include both individual enantiomers and mixtures, racemic or otherwise, thereof. Within the present disclosure, any open valency appearing on a carbon, oxygen, or nitrogen atom in any structure described herein indicates the presence of a hydrogen atom. Where a chiral center exists in a structure, but no specific stereochemistry is shown for that center, both enantiomers, separately or as a mixture, are encompassed by that structure. The methods for the determination of stereochemistry and the separation of stereoisomers are well-known in the art.

The present disclosure is directed to the use of a compound of Formula (III):

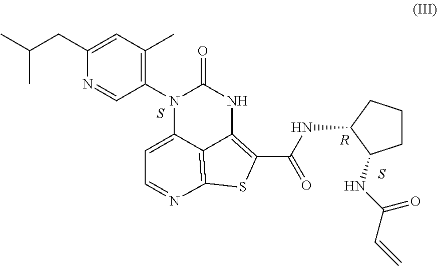

or pharmaceutically acceptable salts, hydrates, polymorphs or solvates thereof. The compound of Formula (III) is also known as N-((1R,2S)-2-Acrylamidocyclopentyl)-5-(S)-(6-isobutyl-4-methylpyridin-3-yl)-4-oxo-4,5-dihydro-3H-1-thia-3,5,8-triazaacenaphthyl ene-2-carboxamide.

The disclosure also relates to methods of using the compounds described herein to treat subjects diagnosed with or suffering from a disease, disorder, or condition mediated by Bruton's tyrosine kinase. These methods are accomplished by administering to the subject a compound of the disclosure in an amount sufficient to inhibit Bruton's tyrosine kinase. In a further aspect, provided herein are methods for inhibiting Bruton's tyrosine kinase in a subject in need of treatment by administering to the subject a composition containing a therapeutically effective amount of the compound of Formula (III).

Some aspects of the disclosure are directed to methods of treating a subject suffering from a malignancy by administering to the subject a composition containing a therapeutically effective amount of the compound of Formula (III). In some aspects, the malignancy is DLBCL. In some aspects, the malignancy is ABC-DLBCL, GCB-DLBCL or non-germinal center B-cell diffuse large B-cell lymphoma (non-GCB-DLBCL). When used for the treatment of DLBCL, the compound of Formula (III) can be administered as a single agent. Alternatively, when used for the treatment of DLBCL, the compound of Formula (III) can be administered in combination with other agents known to be useful for the treatment of DLBCL. When used for the treatment of ABC-DLBCL, GCB-DLBCL or non-GCB-DLBCL, the compound of Formula (III) can be administered as a single agent. Alternatively, when used for the treatment of ABC-DLBCL, germinal center B-cell diffuse large B-cell lymphoma (GCB-DLBCL) or non-germinal center B-cell diffuse large B-cell lymphoma (non-GCB-DLBCL), the compound of Formula (III) can be administered in combination with other agents known to be useful for the treatment of DLBCL.

Other aspects of the disclosure are directed to methods of treating a subject suffering from DLBCL by administering to the subject a composition containing a therapeutically effective amount of a compound of Formula (III). In yet other aspects, compounds of the disclosure can be used to treat ABC-DLBCL, GCB-DLBCL or non-GCB-DLBCL.

DLBCL is the most prevalent type of aggressive non-Hodgkin's lymphoma (NHL) in the United States. Clinical courses of patients with DLBCL are highly heterogeneous. While majority of the patients with DLBCL show response to the initial treatment, approximately one-third of patients have refractory disease or experience relapse after the standard therapies. DLBCL is a clinically and biologically heterogeneous disease, which can be demonstrated by several clinical and molecularly defined prognostic models.

Early and effective treatment of DLBCL is a critical factor affecting the survival of DLBCL patients. The selection of treatment regimens against which DLBCL is resistant delays the onset of effective treatment of the cancer and can lead to growth and spread of the cancer. This, in turn, can have a negative effect on the patient's treatment outcome. Tumor-specific characteristics that are associated with responsiveness to an anti-cancer agent, e.g., a compound of Formula (III), such as the expression of one or more specific genes and/or encoded proteins are useful as a prognostic biomarker for identifying potential patients likely to respond or fail treatment with the compound of Formula (III) at an earlier stage. As a result, patients suffering from DLBCL expressing such a biomarker can be selected for treatment with a compound of Formula (III). In addition, the biomarker can be employed for assessing the response to treatment with a compound of Formula (III).

### Biomarkers Based on Gene Expression Profiling

In certain instances, gene expression profiling (GEP) has been employed for dissecting the molecular heterogeneity and for predicting outcome in DLBCL. GEP can distinguish two prognostic subtypes, GCB-DLBCL and ABC-DLBCL, among whose functional differences include activity of BCR signaling. ABC-DLBCL cells have chronic active BCR signaling, upon which their survival is highly dependent.

BCR signaling is a critical growth and survival pathway in various B cell malignancies, including DLBCL. Upon BCR stimulation, normal and malignant B cells secrete the chemokines CCL3 and CCL4 (MIP-1 α and β) which promotes B cell interactions with accessory cells, such as T helper cells. CCL3 and CCL4 are chemokines of the CC subfamily and inducible in a number of hematopoietic cells, particularly those involved in adaptive immune responses (macrophages, dendritic cells, and B and T lymphocytes). CCL3 signals through the chemokine receptors CCR1 and CCR5, whereas CCL4 signals only through CCR5. CCL3 is a key response gene in B cells, which is up-regulated by BCR signaling, and repressed by Bcl-6. Plasma CCL3 and CCL4 levels are elevated in patients with B-cell malignancies, such as DLBCL and chronic lymphocytic leukemia (CLL). In DLBCL, SCYA3, the gene encoding CCL3, was highly expressed in the ABC subtype in DLBCL. ABC-DLBCL cells, but not GCB cells, secrete high levels of CCL3 and CCL4 after BCR triggering, which was sensitive to inhibition with the BTK inhibotors such as ibrutinib. High CCL3 (>40 pg/ml) serum concentrations have been correlated with a higher international prognostic index (IPI), LDH, and β2 microglobulin, as did CCL4 (>180 pg/ml) with advanced Ann Arbor stages. High CCL3 is correlated with significantly shorter progression-free and overall survival. Further, a correlation between anti-IgM responsiveness in the ABC-DLBL subtype in comparison with the GCB subtype, as well as sensitivity to ibrutinib treatment was observed. Furthermore, in most patients, high concentrations of serum CCL3 and CCL4 returned to low levels after ibrutinib therapy. As aresult of these findings CCL3 and CCL4 protein concentrations can be used as biomarkers for BCR pathway activation and prognosis in DLBCL and assessment of efficacy of BTK inhibitor therapies. Other biomarkers that can also be used as biomarkers for prognosis in DLBCL and assessment of efficacy of BTK inhibitor therapies include biomarker genes selected from ACTG2, LOR, GAPT, CCND2, SELL, GEN1, and HDAC9. These genes encode ACTG2, LOR, GAPT, CCND2, SELL, GEN1, and HDAC9 proteins respectively. ACTG2 (actin, gamma2, smooth muscle, enteric) is ubiquitously expressed highly conserved protein involved in cell motility and maintenance of the cytoskeleton. LOR encodes the protein loricrin, a major protein component of the stratum corneum, the outermost layer of the epidermis. GAPT (GRB2-binding adaptor protein, transmembrane) negatively regulates B cell proliferation following stimulation through the B-cell receptor. CCND2 (cyclin D2) is a regulator of cyclin-dependent kinases and is involved in cell cycle regulation. SELL (selectin L or CD62L) is a cell adhesion molecule found on lymphocytes and is involved in lymphocyte—endothelial cell interactions. GEN1 (Gen endonuclease homolog 1) encodes endonucleases which resolves Holliday junctions during homologous recombination and DNA repair. HDAC9, or histone deacetylase 9, is an enzyme involved in transcriptional regulation, cell cycle progression, and developmental events. Elevated expression of ACTG2, LOR, GAPT, CCND2, SELL, GEN1, HDAC9, or any combination thereof, is indicative of DLBCL. Elevated expression of ACTG2, LOR, GAPT, CCND2, SELL, GEN1, HDAC9, or any combination thereof, is indicative of ABC-DLBCL.

The methods provided herein relate to the use of CCL3, CCL4, ACTG2, LOR, GAPT, CCND2, SELL, GEN1, or HDAC9 expression as a predictive biomarker for identifying responder populations, especially those patients that are likely to be sensitive to treatment with a compound of Formula (III). The methods provided herein provide clinical advantages to the diagnosis and treatment of DLBCL, including easy access to samples, given that CCL3, CCL4, ACTG2, LOR, GAPT, CCND2, SELL, GEN1, HDAC9, or any combination thereof can reliably be quantified in plasma and serum samples, low costs of analyses, and rapid modulation (normalization within days) by therapies targeting the BCR. In some aspects, serum levels of CCL3, CCL4, ACTG2, LOR, GAPT, CCND2, SELL, GEN1, HDAC9, or any combination thereof can be easily quantified by Enzyme-Linked Immunosorbent Assay (ELISA) or other rapid protein detection methods.

Accordingly, in some aspects, patients presenting with higher than normal expression levels of CCL3, CCL4, ACTG2, LOR, GAPT, CCND2, SELL, GEN1, HDAC9, or any combination thereof, are likely to be sensitive to treatment with the compound of Formula (III). In some aspects, patients exhibiting about the same as or lower levels of expression of CCL3, CCL4, ACTG2, LOR, GAPT, CCND2, SELL, GEN1, HDAC9, or any combination thereof, relative to normal are likely to be resistant to treatment with the compound of Formula (III). Thus, measurement of the expression level, gene or protein expression of CCL3, CCL4, ACTG2, LOR, GAPT, CCND2, SELL, GEN1, HDAC9, or any combination thereof is particularly useful to identify patients likely to respond to therapy with the compound of Formula (III).

Some aspects are directed to methods of predicting a positive response of a patient having DLBCL to the compound of Formula (III) by assessing the degree to which the expression of CCL3, CCL4, ACTG2, LOR, GAPT, CCND2, SELL, GEN1, HDAC9, or any combination thereof, decreases following treatment with the compound of Formula (III).

Some aspects are directed to methods and procedures for determining patient sensitivity to the compound of Formula (III). Some aspects are directed to methods for determining or predicting whether an individual requiring therapy for DLBCL will or will not respond to treatment, prior to administration of the treatment, wherein the treatment comprises administration of the compound of Formula (III). Disclosed herein, in certain aspects, are methods for selecting patients diagnosed with DLBCL for treatment with the compound of Formula (III) based on the level of expression the biomarkers CCL3, CCL4, ACTG2, LOR, GAPT, CCND2, SELL, GEN1, HDAC9, or any combination thereof. In some aspects, the methods comprise identifying patient likely to respond to treatment with the compound of Formula (III). In some aspects, the methods comprise determining a treatment regimen. Also disclosed herein, in certain aspects, are methods for evaluating treatment of DLBCL, in an individual in need thereof, comprising: administering to the individual a therapeutically effective amount of the compound of Formula (III) and determining the responsiveness of the patient to treatment based on the level of expression of CCL3, CCL4, ACTG2, LOR, GAPT, CCND2, SELL, GEN1, HDAC9, or any combination thereof. Also disclosed herein, in certain aspects, are methods for treatment of DLBCL, in an individual in need thereof, comprising: administering to the individual a therapeutically effective amount of the compound of Formula (III), determining the responsiveness of the patient to treatment based on the level of expression of CCL3, CCL4, ACTG2, LOR, GAPT, CCND2, SELL, GEN1, HDAC9, or any combination thereof, and continuing treatment if the level of expression of CCL3, CCL4, ACTG2, LOR, GAPT, CCND2, SELL, GEN1, HDAC9, or any combination thereof, has decreased by a predetermined amount. Also disclosed herein, in certain aspects, are methods for treatment of DLBCL or ABC-DLBCL, in an individual in need thereof, comprising: administering to the individual a therapeutically effective amount of the compound of Formula (III), determining the responsiveness of the patient to treatment based on the level of expression of CCL3, CCL4, ACTG2, LOR, GAPT, CCND2, SELL, GEN1, HDAC9, or any combination thereof, and discontinuing treatment if the level of expression of CCL3, CCL4, ACTG2, LOR, GAPT, CCND2, SELL, GEN1, HDAC9, or any combination thereof, has not decreased by a predetermined amount. In some aspects, high levels of expression of CCL3, CCL4, ACTG2, LOR, GAPT, CCND2, SELL, GEN1, HDAC9, or any combination thereof, prior to treatment are predictive of a therapeutic response to treatment with the compound of Formula (III). In some aspects, a decrease in levels of expression (e.g., normalization of expression) of CCL3, CCL4, ACTG2, LOR, GAPT, CCND2, SELL, GEN1, HDAC9, or any combination thereof, following administration of the compound of Formula (III) is predictive of the efficacy of the compound of Formula (III) for treatment of DLBCL or ABC-DLBCL.

In some aspects, methods are provided for treating DLBCL OR ABC-DLBCL by pre-selecting patients who express elevated levels of CCL3, CCL4, ACTG2, LOR, GAPT, CCND2, SELL, GEN1, HDAC9, or any combination thereof, thereby increasing the likelihood of a response, in the patient, to the compound of Formula (III). In some aspects, provided are methods for treating DLBCL or ABC-DLBCL, in a patient in need thereof, by assessing whether the patient expresses elevated levels of CCL3, CCL4, ACTG2, LOR, GAPT, CCND2, SELL, GEN1, HDAC9, or any combination thereof, thereby increasing the likelihood of a response, in the patient, to the compound of Formula (III). In some aspects, provided are methods for treating DLBCL or ABC-DLBCL, in a patient in need thereof, by assessing whether the patient exhibits a change in expression levels of CCL3, CCL4, ACTG2, LOR, GAPT, CCND2, SELL, GEN1, HDAC9, or any combination thereof, following treatment with the compound of Formula (III).

In some aspects, the DLBCL is ABC subtype DLBCL. In some aspects, the Activated B cell-like (ABC) subtype of DLBCL is characterized by a CD79B mutation. In some aspects, the CD79B mutation is a mutation of the immunoreceptor tyrosine-based activation motif (ITAM) signaling module. In some aspects, the CD79B mutation is a missense mutation of the first immunoreceptor tyrosine—based activation motif (ITAM) tyrosine. In some aspects, the CD79B mutation increases surface BCR expression and attenuates Lyn kinase activity. In some aspects, the ABC subtype of DLBCL is characterized by a CD79A mutation. In some aspects, the CD79A mutation is in the immunoreceptor tyrosine-based activation motif (ITAM) signaling module. In some aspects, the CD79A mutation is a splice-donor-site mutation of the ITAM signaling module. In some aspects, the CD79A mutation deletes the ITAM signaling module. In some aspects, the ABC subtype of DLBCL is characterized by a mutation in MyD88, A20, or a combination thereof. In some aspects, the MyD88 mutation is the amino acid substitution L265P in the MYD88 Toll/IL-1 receptor (TIR) domain.

Some aspects are directed to methods for treating DLBCL or ABC-DLBCL in a patient in need thereof comprising: (a) determining an expression level of CCL3, CCL4, ACTG2, LOR, GAPT, CCND2, SELL, GEN1, HDAC9, or any combination thereof, in a sample (e.g., a serum sample) from the patient; and (b) administering, to the patient, a therapeutically effective amount of the compound of Formula (III) if the expression of CCL3, CCL4, ACTG2, LOR, GAPT, CCND2, SELL, GEN1, HDAC9, or any combination thereof, is increased relative to a control or reference level. In some aspects, a reference level is the level of expression of CCL3, CCL4, ACTG2, LOR, GAPT, CCND2, SELL, GEN1, HDAC9, or any combination thereof, in a normal patient (e.g., a patient without DLBCL).

In some aspects, methods are provided for evaluating a response to the compound of Formula (III) in a patient having DLBCL or ABC-DLBCL which comprises determining an expression level of CCL3, CCL4, ACTG2, LOR, GAPT, CCND2, SELL, GEN1, HDAC9, or any combination thereof, in a sample (e.g., a serum sample) from the patient following administration of a therapeutically effective amount of the compound of Formula (III), wherein if a patient sample exhibits a decrease in the levels of expression of CCL3, CCL4, ACTG2, LOR, GAPT, CCND2, SELL, GEN1, HDAC9, or any combination thereof, relative to the control or reference level, a favorable outcome for treatment with the compound of Formula (III) is predicted. In some aspects, methods are provided for evaluating a response to the compound of Formula (III) in a patient having DLBCL or ABC-DLBCL which comprises determining an expression level of CCL3, CCL4, ACTG2, LOR, GAPT, CCND2, SELL, GEN1, HDAC9, or any combination thereof, in a sample (e.g., a serum sample) from the patient following administration of a therapeutically effective amount of the compound of Formula (III), wherein if a patient sample exhibits a decrease in the levels of expression of CCL3, CCL4, ACTG2, LOR, GAPT, CCND2, SELL, GEN1, HDAC9, or any combination thereof, relative to the control or reference level, the patient is characterized as responding to treatment with the compound of Formula (III). In some aspects, a reference level is a level of expression of CCL3, CCL4, ACTG2, LOR, GAPT, CCND2, SELL, GEN1, HDAC9, or any combination thereof, in a normal patient (e.g., a patient without DLBCL). In some aspects, level of CCL3, CCL4, ACTG2, LOR, GAPT, CCND2, SELL, GEN1, HDAC9, or any combination thereof, expression following treatment with the compound of Formula (III) decreases by 5%, 10%, 15%, 20%, 25%, 30%, 35%, 40%, 45%, 50%, 55%, 60%, 65%, 70%, 75%, 80%, 85%, 90%, 95% 99% or greater following treatment with ibrutinib. In some aspects, the level of CCL3 expression following treatment decreases to the level of expression in a normal patient (i.e., normalizes) following treatment with the compound of Formula (III). In some aspects, levels of expression of CCL3, CCL4, ACTG2, LOR, GAPT, CCND2, SELL, GEN1, HDAC9, or any combination thereof, are measured 1 hour, 2 hour, 3 hours, 4 hours, 5 hour, 6 hours, 7 hours, 8 hours, 9 hours, 10 hours, 11 hours, 12 hours, 14 hours, 16 hours, 18 hours, 20 hours, 24 hours, 36 hours, 48 hours, or longer following treatment with the compound of Formula (III).

In some aspects, an exemplary method for treating DLBCL or ABC-DLBCL in a patient in need thereof comprises: (a) determining an expression level of CCL3, CCL4, ACTG2, LOR, GAPT, CCND2, SELL, GEN1, HDAC9, or any combination thereof, in a sample (e.g., a serum sample) from the patient following administration of a therapeutically effective amount of the compound of Formula (III); and (b) modifying, discontinuing, or continuing the treatment based on the expression of CCL3, CCL4, ACTG2, LOR, GAPT, CCND2, SELL, GEN1, HDAC9, or any combination thereof, relative to a control or reference level. In some aspects, a reference level is a level of expression of CCL3, CCL4, ACTG2, LOR, GAPT, CCND2, SELL, GEN1, HDAC9, or any combination thereof, in a sample (e.g., a serum sample) taken from the patient prior to administration of the therapeutically effective amount of the compound of Formula (III). In some aspects, the treatment regimen is continued. In some aspects, the treatment regimen is modified. In some aspects, the dosage of the compound of Formula (III) is increased. In some aspects, the dosage of the compound of Formula (III) is decreased. In some aspects, the dosage of the compound of Formula (III) is not modified. In some aspects, the frequency of administration of the compound of Formula (III) is increased. In some aspects, the frequency of administration of the compound of Formula (III) is decreased. In some aspects, the frequency of administration of the compound of Formula (III) is not modified. In some aspects, the timing of administration of the compound of Formula (III) is modified (e.g., time of day or time relative to administration of other therapeutic agents). In some aspects, the timing of administration of the compound of Formula (III) is not modified. In some aspects, an additional therapeutic agent is administered. In some aspects, an additional anti-cancer agent is administered.

In some aspects, an exemplary method for treating DLBCL or ABC-DLBCL in a patient in need thereof comprises: (a) administering a treatment comprising a therapeutically effective amount of a compound of Formula (III); (b) determining an expression level of CCL3, CCL4, ACTG2, LOR, GAPT, CCND2, SELL, GEN1, HDAC9, or any combination thereof, in a sample from the patient following administration of the treatment; and (c) discontinuing the treatment if the expression of CCL3, CCL4, ACTG2, LOR, GAPT, CCND2, SELL, GEN1, HDAC9, or any combination thereof, is not decreased by a predetermined amount relative to the expression level of CCL3, CCL4, ACTG2, LOR, GAPT, CCND2, SELL, GEN1, HDAC9, or any combination thereof, prior to treatment. In some aspects, the predetermined amount is a decrease in the level of expression of CCL3, CCL4, ACTG2, LOR, GAPT, CCND2, SELL, GEN1, HDAC9, or any combination thereof, by 3% 5%, 10%, 15%, 20%, 25%, 30%, 35%, 40%, 45%, 50%, 55%, 60%, 65%, 70%, 75%, 80%, 85%, 90%, 95%, 99% or greater following treatment with the compound of Formula (III). In some aspects, level of expression of CCL3, CCL4, ACTG2, LOR, GAPT, CCND2, SELL, GEN1, HDAC9, or any combination thereof, is measured 1 hour, 2 hour, 3 hours, 4 hours, 5 hour, 6 hours, 7 hours, 8 hours, 9 hours, 10 hours, 11 hours, 12 hours, 14 hours, 16 hours, 18 hours, 20 hours, 24 hours, 36 hours, 48 hours, or longer following treatment with a compound of Formula (III).

In some aspects, methods are provided for predicting a response to a compound of Formula (III) in a patient having DLBCL or ABC-DLBCL which comprises determining an expression level of CCL3, CCL4, ACTG2, LOR, GAPT, CCND2, SELL, GEN1, HDAC9, or any combination thereof, in a sample (e.g., a serum sample) from the patient prior to administering the compound of Formula (III) and comparing it to a control or reference, wherein if a patient sample exhibits high levels of expression of CCL3, CCL4, ACTG2, LOR, GAPT, CCND2, SELL, GEN1, HDAC9, or any combination thereof, relative to the control or reference level, a favorable outcome for treatment with a compound of Formula (III) is predicted. In some aspects, a reference level is a level of expression of CCL3, CCL4, ACTG2, LOR, GAPT, CCND2, SELL, GEN1, HDAC9, or any combination thereof, in a normal patient (e.g., a patient without DLBCL).

In some aspects, methods are provided for treating DLBCL or ABC-DLBCL in a selected patient comprising administering to the selected patient a therapeutically effective amount of a compound of Formula (III) in an amount effective to treat DLBCL or ABC-DLBCL, wherein said selected patient has a high expression level of CCL3, CCL4, ACTG2, LOR, GAPT, CCND2, SELL, GEN1, HDAC9, or any combination thereof, prior to administration of the compound of Formula (III) and wherein high expression levels of CCL3, CCL4, ACTG2, LOR, GAPT, CCND2, SELL, GEN1, HDAC9, or any combination thereof, relative to normal indicates that the selected patient would benefit from continued treatment with the compound of Formula (III).

In some aspects, methods are provided for identifying a patient that is likely to respond therapeutically to treatment with a compound of Formula (III), wherein the method comprises: (a), measuring in a sample (e.g., a serum sample) obtained from a patient having DLBCL or ABC-DLBCL or is suspected of having DLBCL or ABC-DLBCL the expression level of CCL3, CCL4, ACTG2, LOR, GAPT, CCND2, SELL, GEN1, HDAC9, or any combination thereof; (b) comparing the level obtained in step (a) to the level of expression of said CCL3, CCL4, ACTG2, LOR, GAPT, CCND2, SELL, GEN1, HDAC9, or any combination thereof, in a control sample, wherein an increase in the level of CCL3, CCL4, ACTG2, LOR, GAPT, CCND2, SELL, GEN1, HDAC9, or any combination thereof, measured in step (a) relative to the control sample indicates that the patient will respond therapeutically to treatment with the compound of Formula (III), whereas the level of CCL3, CCL4, ACTG2, LOR, GAPT, CCND2, SELL, GEN1, HDAC9, or any combination thereof, that is not increased or is decreased relative to the control sample indicates that the patient is not likely to respond to treatment or be resistant to treatment with the compound of Formula (III).

In some aspects, methods are provided for predicting whether a patient will respond therapeutically to a method of treating DLBCL or ABC-DLBCL comprising administering a compound of Formula (III), wherein the method comprises: (a) measuring in a sample (e.g., a serum sample) obtained from the patient the expression level of CCL3, CCL4, ACTG2, LOR, GAPT, CCND2, SELL, GEN1, HDAC9, or any combination thereof; (b) comparing the level obtained in step (a) to the level of expression of said CCL3, CCL4, ACTG2, LOR, GAPT, CCND2, SELL, GEN1, HDAC9, or any combination thereof, in a control sample, wherein an increase in the level of CCL3, CCL4, ACTG2, LOR, GAPT, CCND2, SELL, GEN1, HDAC9, or any combination thereof, measured in step (a) indicates that the patient will respond therapeutically to treatment with the compound of Formula (III), whereas the level of CCL3, CCL4, ACTG2, LOR, GAPT, CCND2, SELL, GEN1, HDAC9, or any combination thereof, that is not increased or is decreased relative to the control sample indicates that the patient is not likely to respond to treatment or be resistant to treatment with the compound of Formula (III).

In some aspects, the methods provided herein are practiced iteratively over time, wherein decreased levels of CCL3, CCL4, ACTG2, LOR, GAPT, CCND2, SELL, GEN1, HDAC9, or any combination thereof, in the patient sample relative to a pretreatment or reference sample suggest a favorable response of a patient to treatment with a compound of Formula (III) and levels of CCL3, CCL4, ACTG2, LOR, GAPT, CCND2, SELL, GEN1, HDAC9, or any combination thereof, in the patient sample that are increased relative to the control sample indicates that the patient is not likely to respond to treatment or be resistant to treatment with the compound of Formula (III). Accordingly, in some aspects, methods are provided of monitoring the treatment of a patient having DLBCL or ABC-DLBCL, wherein the DLBCL or ABC-DLBCL is treated by a method comprising administering the compound of Formula (III) to the patient alone or in combination with an anti-cancer agent or a neoplastic agent.

In some aspects, level of expression of CCL3, CCL4, ACTG2, LOR, GAPT, CCND2, SELL, GEN1, HDAC9, or any combination thereof, is measured 1 hour, 2 hour, 3 hours, 4 hours, 5 hour, 6 hours, 7 hours, 8 hours, 9 hours, 10 hours, 11 hours, 12 hours, 14 hours, 16 hours, 18 hours, 20 hours, 24 hours, 36 hours, 48 hours, or longer following treatment with a compound of Formula (III). In some aspects, the expression level of CCL3, CCL4, ACTG2, LOR, GAPT, CCND2, SELL, GEN1, HDAC9, or any combination thereof, is measured 1 hour, 2 hour, 3 hours, 4 hours, 5 hour, 6 hours, 7 hours, 8 hours, 9 hours, 10 hours, 11 hours, 12 hours, 14 hours, 16 hours, 18 hours, 20 hours, 24 hours, 36 hours, 48 hours, or longer following a single dosage with a compound of Formula (III). In some aspects, the expression level of CCL3, CCL4, ACTG2, LOR, GAPT, CCND2, SELL, GEN1, HDAC9, or any combination thereof, is measured 1 hour, 2 hour, 3 hours, 4 hours, 5 hour, 6 hours, 7 hours, 8 hours, 9 hours, 10 hours, 11 hours, 12 hours, 14 hours, 16 hours, 18 hours, 20 hours, 24 hours, 36 hours, 48 hours, or longer following a multiple administrations with a compound of Formula (III). In some aspects, the expression level of CCL3, CCL4, ACTG2, LOR, GAPT, CCND2, SELL, GEN1, HDAC9, or any combination thereof, is measured 1 hour, 2 hour, 3 hours, 4 hours, 5 hour, 6 hours, 7 hours, 8 hours, 9 hours, 10 hours, 11 hours, 12 hours, 14 hours, 16 hours, 18 hours, 20 hours, 24 hours, 36 hours, 48 hours, or longer following a multiple administrations with a compound of Formula (III) and after the last dosage. In some aspects, the expression level of CCL3, CCL4, ACTG2, LOR, GAPT, CCND2, SELL, GEN1, HDAC9, or any combination thereof, is measured 1 hour, 2 hour, 3 hours, 4 hours, 5 hour, 6 hours, 7 hours, 8 hours, 9 hours, 10 hours, 11 hours, 12 hours, 14 hours, 16 hours, 18 hours, 20 hours, 24 hours, 36 hours, 48 hours, or longer following administration of the last dosage a compound of Formula (III) in a treatment regimen.

In some aspects, the expression level of CCL3, CCL4, ACTG2, LOR, GAPT, CCND2, SELL, GEN1, HDAC9, or any combination thereof, is monitored over time during the course of a treatment regimen with a compound of Formula (III). In some aspects, the expression level of CCL3, CCL4, ACTG2, LOR, GAPT, CCND2, SELL, GEN1, HDAC9, or any combination thereof, is measured every day, every 2 days, every 3 days, every 4 days, every 5 days, every 6 days, every week, every 2 weeks, every 3 weeks, every month or longer interval. In some aspects, the expression level of CCL3, CCL4, ACTG2, LOR, GAPT, CCND2, SELL, GEN1, HDAC9, or any combination thereof, is monitored over time during the course of a treatment regimen with a compound of Formula (III) where the expression level of CCL3, CCL4, ACTG2, LOR, GAPT, CCND2, SELL, GEN1, HDAC9, or any combination thereof, is determined following each administration of the compound of Formula (III). In some aspects, the expression level of CCL3, CCL4, ACTG2, LOR, GAPT, CCND2, SELL, GEN1, HDAC9, or any combination thereof, is monitored over time during the course of a treatment regimen with a compound of Formula (III) where the expression level of CCL3, CCL4, ACTG2, LOR, GAPT, CCND2, SELL, GEN1, HDAC9, or any combination thereof, is determined following multiple administrations of the compound of Formula (III). In some aspects, the expression level of CCL3, CCL4, ACTG2, LOR, GAPT, CCND2, SELL, GEN1, HDAC9, or any combination thereof, is monitored over time during the course of a treatment regimen with a compound of Formula (III) where the expression level of CCL3, CCL4, ACTG2, LOR, GAPT, CCND2, SELL, GEN1, HDAC9, or any combination thereof, is determined following 2, 3, 4, 5, 6, 7, 8, 9, 10, 11, 12 or more administrations of the compound of Formula (III).

In some aspects, the expression level of CCL3, CCL4, ACTG2, LOR, GAPT, CCND2, SELL, GEN1, HDAC9, or any combination thereof, is monitored over time during the course of a treatment regimen with a compound of Formula (III), wherein an increase in the expression level of CCL3, CCL4, ACTG2, LOR, GAPT, CCND2, SELL, GEN1, HDAC9, or any combination thereof, indicates that the patient is resistant or will become resistant to treatment with the compound of Formula (III). In some aspects, the expression level of CCL3, CCL4, ACTG2, LOR, GAPT, CCND2, SELL, GEN1, HDAC9, or any combination thereof, is monitored over time during the course of a treatment regimen with a compound of Formula (III), wherein if a change in the expression level of CCL3, CCL4, ACTG2, LOR, GAPT, CCND2, SELL, GEN1, HDAC9, or any combination thereof, is detected relative to a reference level, the treatment regimen is modified, continued or discontinued. In some aspects, the treatment regimen is discontinued. In some aspects, the treatment regimen is continued. In some aspects, the treatment regimen is modified. In some aspects, the dosage of the compound of Formula (III) is increased. In some aspects, the dosage of the compound of Formula (III) is decreased. In some aspects, the dosage of the compound of Formula (III) not modified. In some aspects, the frequency of administration of the compound of Formula (III) is increased. In some aspects, the frequency of administration of the compound of Formula (III) is decreased. In some aspects, the frequency of administration of the compound of Formula (III) is not modified. In some aspects, the timing of administration of the compound of Formula (III) is modified (e.g., time of day or time relative to administration of other therapeutic agents). In some aspects, the timing of administration of the compound of Formula (III) is not modified. In some aspects, an additional therapeutic agent is administered.

In some aspects, where the expression level of CCL3, CCL4, ACTG2, LOR, GAPT, CCND2, SELL, GEN1, HDAC9, or any combination thereof, is increased over the course of therapy with a compound of Formula (III), the dosage of the compound of Formula (III) is increased. In some aspects, where the expression level of CCL3, CCL4, ACTG2, LOR, GAPT, CCND2, SELL, GEN1, HDAC9, or any combination thereof, is increased over the course of therapy, the frequency of administration of the compound of Formula (III) is increased. In some aspects, where the expression level of CCL3, CCL4, ACTG2, LOR, GAPT, CCND2, SELL, GEN1, HDAC9, or any combination thereof, is increased over the course of therapy, an additional therapeutic agent is administered.

In some aspects, “high expression levels” of CCL3, CCL4, ACTG2, LOR, GAPT, CCND2, SELL, GEN1, HDAC9, or any combination thereof, in a patient relative to normal means that the patient exhibits a 1.5-fold, 2-fold, 3-fold, 4-fold, 5-fold, 6-fold, 7-fold, 8-fold, 9-fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, or higher increase in the pretien expression levels of CCL3, CCL4, ACTG2, LOR, GAPT, CCND2, SELL, GEN1, HDAC9, or any combination thereof. In some aspects, “high expression levels” of CCL3, CCL4, ACTG2, LOR, GAPT, CCND2, SELL, GEN1, HDAC9, or any combination thereof, in a patient relative to normal means that the patient exhibits a 1.5-fold, 2-fold, 3-fold, 4-fold, 5-fold, 6-fold, 7-fold, 8-fold, 9-fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, or higher increase in the expression of nucleic acid (e.g., mRNA) encoding CCL3, CCL4, ACTG2, LOR, GAPT, CCND2, SELL, GEN1, HDAC9, or any combination thereof.

In some aspects, the method comprises obtaining a sample (e.g., a serum sample) from the patient and measuring the protein expression levels of CCL3, CCL4, ACTG2, LOR, GAPT, CCND2, SELL, GEN1, HDAC9, or any combination thereof, protein. In some aspects, measuring the protein expression levels of CCL3, CCL4, ACTG2, LOR, GAPT, CCND2, SELL, GEN1, HDAC9, or any combination thereof, comprises an immunoassay. In some aspects, measuring the protein expression levels of CCL3, CCL4, ACTG2, LOR, GAPT, CCND2, SELL, GEN1, HDAC9, or any combination thereof, comprises an ELISA. In some aspects, measuring the protein expression levels of CCL3, CCL4, ACTG2, LOR, GAPT, CCND2, SELL, GEN1, HDAC9, or any combination thereof, comprises detecting CCL3, CCL4, ACTG2, LOR, GAPT, CCND2, SELL, GEN1, HDAC9, or any combination thereof, protein with an antibody. In some aspects, the antibody is labeled. In some aspects, measuring the protein expression levels of CCL3, CCL4, ACTG2, LOR, GAPT, CCND2, SELL, GEN1, HDAC9, or any combination thereof, comprises detecting CCL3, CCL4, ACTG2, LOR, GAPT, CCND2, SELL, GEN1, HDAC9, or any combination thereof, with a first antibody to form an antibody complex with CCL3, CCL4, ACTG2, LOR, GAPT, CCND2, SELL, GEN1, HDAC9, or any combination thereof, and then detecting the antibody complex with a secondary antibody that binds to the first antibody. In some aspects, the antibody is labeled.

In some aspects, the method comprises obtaining a sample containing nucleic acid from the patient and measuring the expression level of the nucleic acid encoding CCL3, CCL4, ACTG2, LOR, GAPT, CCND2, SELL, GEN1, HDAC9, or any combination thereof. In some aspects, the method comprises isolating or purifying mRNA from the sample. In some aspects, the method comprises amplifying the mR A transcripts, e.g., by RT-PCR. In some aspects, a higher baseline level of CCL3, CCL4, ACTG2, LOR, GAPT, CCND2, SELL, GEN1, HDAC9, or any combination thereof, (as assessed by, e.g., determining the cycle number at which the fluorescence passes the set threshold level (“ct”) mRNA expression of CCL3, CCL4, ACTG2, LOR, GAPT, CCND2, SELL, GEN1, HDAC9, or any combination thereof,) indicates a higher likelihood that the cancer will be sensitive to treatment with the compound of Formula (III).

In some aspects, the level of expression of CCL3, CCL4, ACTG2, LOR, GAPT, CCND2, SELL, GEN1, HDAC9, or any combination thereof, in a sample is compared to the level of expression in a reference DLBCL or ABC-DLBCL cell or population of DLBCL or ABC-DLBCL cells. In some aspects, the level of expression of CCL3, CCL4, ACTG2, LOR, GAPT, CCND2, SELL, GEN1, HDAC9, or any combination thereof, in a sample is compared to the level of expression in a reference DLBCL or ABC-DLBCL cell line. In some aspects, the level of expression of CCL3, CCL4, ACTG2, LOR, GAPT, CCND2, SELL, GEN1, HDAC9, or any combination thereof, in a sample is compared to the level of expression in a reference DLBCL cell or population of DLBCL cells that is known to be resistant to treatment with a compound of Formula (III). In some aspects, the level of expression of CCL3, CCL4, ACTG2, LOR, GAPT, CCND2, SELL, GEN1, HDAC9, or any combination thereof, in a sample is compared to the level of expression in a reference DLBCL cell or population of DLBCL cells that is known to be sensitive to treatment with a compound of Formula (III). In some aspects, the level of expression of CCL3, CCL4, ACTG2, LOR, GAPT, CCND2, SELL, GEN1, HDAC9, or any combination thereof, in a sample is compared to the level of expression in a reference DLBCL cell line that is known to be resistant to treatment with a compound of Formula (III). In some aspects, the level of expression of CCL3, CCL4, ACTG2, LOR, GAPT, CCND2, SELL, GEN1, HDAC9, or any combination thereof, in a sample is compared to the level of expression in a reference DLBCL cell line that is known to be sensitive to treatment with a compound of Formula (III). In some aspects, the DLBCL cell line is an activated B-cell-like (ABC)-DLBCL cell line. In some aspects, the DLBCL cell line is a germinal center B-cell-like (GCB)-DLBCL cell line. In some aspects, the DLBCL cell line is OCI-Ly1, OCI-Ly2, OCI-Ly3, OCI-Ly4, OCI-Ly6, OCI-Ly7, OCI-Ly1O, OCI-Ly18, OCI-Ly19, U2932, DB, HBL-1, RIVA, or TMD8. In some aspects, the DLBCL cell line sensitive to treatment with a compound of Formula (III) is TMD8, HBL-1 or OCI-Ly10. In some aspects, the DLBCL cell line resistant to treatment with a compound of Formula (III) is OCI-Ly3, DB or OCI-Ly19.

Some aspects are directed to methods wherein a subject having DLBCL is treated with a therapeutically effective amount of a compound of Formula (III) for longer than six months and after about six months of treatment, the subject is monitored at predetermined intervals of time to determine level of expression of CCL3 and/or CCL4. In some aspects, monitoring comprises: (a) determining an expression level of CCL3, CCL4, ACTG2, LOR, GAPT, CCND2, SELL, GEN1, HDAC9, or any combination thereof, in a sample (e.g., a serum sample) following or during the course of treatment with a compound of Formula (III); and (b) modifying or continuing the treatment based on the expression of CCL3, CCL4, ACTG2, LOR, GAPT, CCND2, SELL, GEN1, HDAC9, or any combination thereof, relative to a control or reference level. In some aspects, a reference level is a level of expression of CCL3, CCL4, ACTG2, LOR, GAPT, CCND2, SELL, GEN1, HDAC9, or any combination thereof, in a sample (e.g., a serum sample) taken from the patient prior to administration of the therapeutically effective amount of the compound of Formula (III). In some aspects, a reference level is the level of expression of CCL3, CCL4, ACTG2, LOR, GAPT, CCND2, SELL, GEN1, HDAC9, or any combination thereof, in a normal patient (e.g., a patient without DLBCL). In some aspects, the treatment regimen is continued. In some aspects, the treatment regimen is modified. In some aspects, the dosage of the compound of Formula (III) is increased. In some aspects, the dosage of the compound of Formula (III) is decreased. In some aspects, the dosage of the compound of Formula (III) not modified. In some aspects, the frequency of administration of the compound of Formula (III) is increased. In some aspects, the frequency of administration of the compound of Formula (III) is decreased. In some aspects, the frequency of administration of the compound of Formula (III) is not modified. In some aspects, the timing of administration of the compound of Formula (III) is modified (e.g., time of day or time relative to administration of other therapeutic agents). In some aspects, the timing of administration of the compound of Formula (III) is not modified. In some aspects, an additional therapeutic agent is administered. In some aspects, an additional anti-cancer agent is administered.

In some aspects, the method further comprises discontinuing treatment with a compound of Formula (III) if level of expression of CCL3, CCL4, ACTG2, LOR, GAPT, CCND2, SELL, GEN1, HDAC9, or any combination thereof, increase relative to a reference or control over the course of treatment with the compound of Formula (III). In some aspects, the method further comprises continuing treatment with a compound of Formula (III) if level of expression of CCL3, CCL4, ACTG2, LOR, GAPT, CCND2, SELL, GEN1, HDAC9, or any combination thereof, decreases relative to a reference level. In some aspects, the reference is level of expression of CCL3, CCL4, ACTG2, LOR, GAPT, CCND2, SELL, GEN1, HDAC9, or any combination thereof, in a DLBCL patient. In some aspects, the reference is level of expression of CCL3, CCL4, ACTG2, LOR, GAPT, CCND2, SELL, GEN1, HDAC9, or any combination thereof, in the same patient prior to treatment with a compound of Formula (III).

In some aspects, an individual is characterized as having a DLBCL if the individual shows an increase in expression level in at least one biomarker selected from CCL3, CCL4, ACTG2, LOR, GAPT, CCND2, SELL, GEN1, HDAC9, or any combination thereof, relative to a control.

In some aspects, also disclosed herein are methods of assessing an individual having DLBCL or ABC-DLBCL for treatment with the compound of Formula (III) by determining the expression level of at least one biomarker selected from CCL3, CCL4, ACTG2, LOR, GAPT, CCND2, SELL, GEN1, HDAC9, or any combination thereof, and administer to the individual a therapeutically effective amount of the compound of Formula (III) if there is an increase in expression level in at least one biomarker selected from CCL3, CCL4, ACTG2, LOR, GAPT, CCND2, SELL, GEN1, HDAC9, or any combination thereof.

In some aspects, further disclosed herein are methods of monitoring the disease progression in an individual having DLBCL or ABC-DLBCL by determining the expression level of at least one biomarker selected from CCL3, CCL4, ACTG2, LOR, GAPT, CCND2, SELL, GEN1, HDAC9, or any combination thereof; and characterize the individual as having DLBCL or ABC-DLBCL if the individual shows an increase in expression level in at least one biomarker CCL3, CCL4, ACTG2, LOR, GAPT, CCND2, SELL, GEN1, HDAC9, or any combination thereof.

The methods provided herein relate to the use of CD10, BCL6, or MUM1 expression as a predictive biomarker for identifying responder populations, especially those patients that are likely to be sensitive to treatment with a compound of Formula (III) such as the compound of Formula (III). The methods provided herein provide clinical advantages to the diagnosis and treatment of GCB-DLBCL, including easy access to samples, given that CD10, BCL6, MUM1, or any combination thereof can reliably be quantified in plasma and serum samples, low costs of analyses, and rapid modulation (normalization within days) by therapies targeting the BCR. In some aspects, serum levels of CD10, BCL6, MUM1 or any combination thereof, can be easily quantified by Enzyme-Linked Immunosorbent Assay (ELISA) or other rapid protein detection methods. Other methods for detecting CD10, BCL6, MUM1 or any combination thereof can be found in Hans et. Al. (Confirmation of the molecular classification of diffuse large B-cell lymphoma by immunohistochemistry using a tissue microarray; Neoplasia (2004) 103: 275-282) which is hereby incorporated in its entirety. Accordingly, in some aspects, patients presenting with higher than normal expression levels of CD10, BCL6, or a combination thereof, and lower than normal expression on MUM1 are likely to be sensitive to treatment with the compound of Formula (III). In some aspects, patients exhibiting about the same as or lower levels of expression of CD10, BCL, or a combination thereof, and higher than normal expression of MUM1, relative to normal are likely to be resistant to treatment with the compound of Formula (III). Thus, measurement of the expression level, gene or protein expression of CD10, BCL6, MUM1, or a combination thereof is particularly useful to identify patients likely to respond to therapy with the compound of Formula (III).

Some aspects are directed to methods and procedures for determining patient sensitivity to the compound of Formula (III). Some aspects are directed to methods for determining or predicting whether an individual requiring therapy for GCB-DLBCL will or will not respond to treatment, prior to administration of the treatment, wherein the treatment comprises administration of the compound of Formula (III). Disclosed herein, in certain aspects, are methods for selecting patients diagnosed with diffuse large GCB-DLBCL for treatment with the compound of Formula (III) based on the level of expression the biomarkers CD10, BCL6, MUM1, or any combination thereof. In some aspects, the methods comprise identifying a patient likely to respond to treatment with the compound of Formula (III). In some aspects, the methods comprise determining a treatment regimen. Also disclosed herein, in certain aspects, are methods for evaluating treatment of GCB-DLBCL, in an individual in need thereof, comprising: administering to the individual a therapeutically effective amount of the compound of Formula (III) and determining the responsiveness of the patient to treatment based on the level of expression of CD10, BCL6, MUM1, or any combination thereof. In some aspects, high levels of expression of CD10, BCL6, or a combination thereof and low levels of expression of MUM1 prior to treatment are predictive of a therapeutic response to treatment with the compound of Formula (III). In some aspects, methods are provided for treating GCB-DLBCL by pre-selecting patients who express elevated levels of CD10, BCL6, or a combination thereof, and low levels of MUM1, thereby increasing the likelihood of a response, in the patient, to the compound of Formula (III). In some aspects, provided are methods for treating GCB-DLBCL, in a patient in need thereof, by assessing whether the patient expresses elevated levels of CD10, BCL6, or a combination thereof, and low levels of MUM1, thereby increasing the likelihood of a response, in the patient, to the compound of Formula (III). Some aspects are directed to methods for treating GCB-DLBCL in a patient in need thereof comprising: (a) determining an expression level of CD10, BCL6, MUM1 or any combination thereof, in a sample (e.g., a serum sample) from the patient; and (b) administering, to the patient, a therapeutically effective amount of the compound of Formula (III) if the expression of CD10, BCL6, or a combination thereof, is increased relative to a control or reference level and expression of MUM1 is decreased relative to a control or reference level. In some aspects, a reference level is a level of expression of CD10, BCL6, MUM1 or any combination thereof, in a normal patient (e.g., a patient without DLBCL, or GCB-DLBCL).

In some aspects, an exemplary method for treating GCB-DLBCL in a patient in need thereof comprises: (a) determining an expression level of CD10, BCL6, MUM1 or any combination thereof, in a sample (e.g., a serum sample) from the patient following administration of a therapeutically effective amount of the compound of Formula (III); and (b) modifying, discontinuing, or continuing the treatment based on the expression of CD10, BCL6, MUM1 or any combination thereof, relative to a control or reference level. In some aspects, a reference level is the level of expression of CD10, BCL6, MUM1, or any combination thereof, in a sample (e.g., a serum sample) taken from the patient prior to administration of the therapeutically effective amount of the compound of Formula (III). In some aspects, the treatment regimen is continued. In some aspects, the treatment regimen is modified. In some aspects, the dosage of the compound of Formula (III) is increased. In some aspects, the dosage of the compound of Formula (III) is decreased. In some aspects, the dosage of the compound of Formula (III) is not modified. In some aspects, the frequency of administration of the compound of Formula (III) is increased. In some aspects, the frequency of administration of the compound of Formula (III) is decreased. In some aspects, the frequency of administration of the compound of Formula (III) is not modified. In some aspects, the timing of administration of the compound of Formula (III) is modified (e.g., time of day or time relative to administration of other therapeutic agents). In some aspects, the timing of administration of the compound of Formula (III) is not modified. In some aspects, an additional therapeutic agent is administered. In some aspects, an additional anti-cancer agent is administered.

In some aspects, methods are provided for predicting a response to a compound of Formula (III) in a patient having GCB-DLBCL which comprises determining an expression level of CD10, BCL6, MUM1 or any combination thereof, in a sample (e.g., a serum sample) from the patient prior to administering the compound of Formula (III) and comparing it to a control or reference, wherein if a patient sample exhibits high levels of expression of CD10, BCL6, MUM1 or any combination thereof, relative to the control or reference level, a favorable outcome for treatment with a compound of Formula (III) is predicted. In some aspects, a reference level is a level of expression of CD10, BCL6, MUM1 or any combination thereof, in a normal patient (e.g., a patient without DLBCL or GCB-BLBCL).

In some aspects, methods are provided for treating GCB-DLBCL in a selected patient comprising administering to the selected patient a therapeutically effective amount of a compound of Formula (III) in an amount effective to treat GCB-DLBCL, wherein said selected patient has a high expression level of CD10, BCL6, or a combination thereof, and low expression levels of MUM1, prior to administration of the compound of Formula (III) and wherein high expression levels of CD10, BCL6, or a combination thereof, and low levels of MUM1, relative to normal indicates that the selected patient would benefit from continued treatment with the compound of Formula (III).

In some aspects, methods are provided for identifying a patient that is likely to respond therapeutically to treatment with a compound of Formula (III), wherein the method comprises: (a), measuring in a sample (e.g., a serum sample) obtained from a patient having GCB-DLBCL or is suspected of having GCB-DLBCL the expression level of CD10, BCL6, MUM1 or any combination thereof; (b) comparing the level obtained in step (a) to the level of expression of said CD10, BCL6, MUM1 or any combination thereof, in a control sample, wherein an increase in the level of CD10, BCL6, or a combination thereof, and a decrease in the level of MUM1 measured in step (a) relative to the control sample indicates that the patient will respond therapeutically to treatment with the compound of Formula (III).

In some aspects, methods are provided for predicting whether a patient will respond therapeutically to a method of treating GCB-DLBCL comprising administering a compound of Formula (III), wherein the method comprises: (a) measuring in a sample (e.g., a serum sample) obtained from the patient the expression level of CD10, BCL6, MUM1, or any combination thereof; (b) comparing the level obtained in step (a) to the level of expression of said CD10, BCL6, MUM1, or any combination thereof, in a control sample, wherein an increase in the level of CD10, BCL6, or a combination thereof, and a decrease in MUM1 expression measured in step (a) indicates that the patient will respond therapeutically to treatment with the compound of Formula (III),.

In some aspects, “high expression levels” of CD10, BCL6, or a combination thereof, in a patient relative to normal means that the patient exhibits a 1.5-fold, 2-fold, 3-fold, 4-fold, 5-fold, 6-fold, 7-fold, 8-fold, 9-fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, or higher increase in the protein expression levels of CD10, BCL6, or a combination thereof. In some aspects, “high expression levels” of CD10, BCL6, or a combination thereof, in a patient relative to normal means that the patient exhibits a 1.5-fold, 2-fold, 3-fold, 4-fold, 5-fold, 6-fold, 7-fold, 8-fold, 9-fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, or higher increase in the expression of nucleic acid (e.g., mRNA) encoding CD10, BCL6, MUM1 or any combination thereof. In some aspects, “low expression levels” of MUM1, in a patient relative to normal means that the patient exhibits a 1.5-fold, 2-fold, 3-fold, 4-fold, 5-fold, 6-fold, 7-fold, 8-fold, 9-fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, or higher decrease in the protein expression levels of MUM1. In some aspects, “low expression levels” of MUM1, in a patient relative to normal means that the patient exhibits a 1.5-fold, 2-fold, 3-fold, 4-fold, 5-fold, 6-fold, 7-fold, 8-fold, 9-fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, or higher decrease in the expression of nucleic acid (e.g., mRNA) encoding.

In some aspects, the method comprises obtaining a sample (e.g., a serum sample) from the patient and measuring the protein expression levels of CD10, BCL6, MUM1 or any combination thereof, protein. In some aspects, measuring the protein expression levels of CD10, BCL6, MUM1 or any combination thereof, comprises an immunoassay. In some aspects, measuring the protein expression levels of CD10, BCL6, MUM1 or any combination thereof, comprises an ELISA. In some aspects, measuring the protein expression levels of CD10, BCL6, MUM1 or any combination thereof, comprises detecting protein levels of CD10, BCL6, MUM1 or any combination thereof, with an antibody. In some aspects, the antibody is labeled. In some aspects, measuring the protein expression levels of CD10, BCL6, MUM1 or any combination thereof, comprises detecting CD10, BCL6, MUM1 or any combination thereof, with a first antibody to form an antibody complex with CD10, BCL6, MUM1 or any combination thereof, and then detecting the antibody complex with a secondary antibody that binds to the first antibody. In some aspects, the antibody is labeled.

In some aspects, the method comprises obtaining a sample containing nucleic acid from the patient and measuring the expression level of the nucleic acid encoding CD10, BCL6, MUM1 or any combination thereof. In some aspects, the method comprises isolating or purifying mRNA from the sample. In some aspects, the method comprises amplifying the mRNA transcripts, e.g., by RT-PCR. In some aspects, a higher baseline level of CD10, BCL6, MUM1 or any combination thereof, (as assessed by, e.g., determining the cycle number at which the fluorescence passes the set threshold level (“ct”) mRNA expression of CD10, BCL6, MUM1 or any combination thereof,) indicates a higher likelihood that the cancer will be sensitive to treatment with the compound of Formula (III).

In some aspects, the level of expression of CD10, BCL6, MUM1 or any combination thereof, in a sample is compared to the level of expression in a reference GCB-DLBCL cell or population of GCB-DLBCL cells. In some aspects, the level of expression of CD10, BCL6, MUM1 or any combination thereof, in a sample is compared to the level of expression in a reference GCB-DLBCL cell line. In some aspects, the level of expression of CD10, BCL6, MUM1 or any combination thereof, in a sample is compared to the level of expression in a reference GCB-DLBCL cell or population of GCB-DLBCL cells that is known to be resistant to treatment with a compound of Formula (III). In some aspects, the level of expression of CD10, BCL6, MUM1 or any combination thereof, in a sample is compared to the level of expression in a reference GCB-DLBCL cell or population of GCB-DLBCL cells that is known to be sensitive to treatment with a compound of Formula (III). In some aspects, the level of expression of CD10, BCL6, MUM1 or any combination thereof, in a sample is compared to the level of expression in a reference DLBCL cell line that is known to be resistant to treatment with a compound of Formula (III). In some aspects, the level of expression of CD10, BCL6, MUM1 or any combination thereof, in a sample is compared to the level of expression in a reference GCB-DLBCL cell line that is known to be sensitive to treatment with a compound of Formula (III). In some aspects, the GCB-DLBCL or DLBCL cell line is OCI-Ly1, OCI-Ly2, OCI-Ly3, OCI-Ly4, OCI-Ly6, OCI-Ly7, OCI-Ly1O, OCI-Ly18, OCI-Ly19, U2932, DB, HBL-1, RIVA, or TMD8. In some aspects, the GCB-DLBCL or DLBCL cell line sensitive to treatment with a compound of Formula (III) is TMD8, HBL-1 or OCI-Ly10. In some aspects, the GCB-DLBCL or DLBCL cell line resistant to treatment with a compound of Formula (III) is OCI-Ly3, DB or OCI-Ly19.

In some aspects, maintenance therapy comprises administration of a daily dosage of a compound of Formula (III). For use in the diagnostic and therapeutic applications described herein, kits and articles of manufacture are also described herein. Such kits can comprise a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. The containers are formed from any acceptable material including, e.g., glass or plastic. In some aspects, the kits provided herein are for use in determining the level of expression of CD10, BCL6, MUM1 or a combination thereof. In some aspects, the kits provided herein are for use as a companion diagnostic with a compound of Formula (III). In some aspects the kits are employed for selecting patients for treatment with a compound of Formula (III), for identifying subjects as sensitive to a compound of Formula (III) of for evaluating treatment with a compound of Formula (III). In some aspects the kits are employed for selecting patients for treatment with a compound of Formula (III), for identifying subjects as resistant or likely to become resistant to a compound of Formula (III), for monitoring the development of resistance to a compound of Formula (III), or combinations thereof.

The kits provided herein contain one or more reagents for the detection of CD10, BCL6, MUM1 or any combination thereof, expression. Exemplary reagents include but are not limited to, antibodies, buffers, nucleic acids, microarrays, ELISA plates, substrates for enzymatic staining, chromagens or other materials, such as slides, containers, microtiter plates, and optionally, instructions for performing the methods. Those of skill in the art will recognize many other possible containers and plates and reagents that can be used for contacting the various materials.

In some aspects, an individual is characterized as having GCB-DLBCL if the individual shows an increase in expression level in at least one biomarker selected from CD10, BCL6, or a combination thereof and optionally lover levels of MUM1, relative to a control.

In some aspects, also disclosed herein are methods of assessing an individual having GCB-DLBCL for treatment with the compound of Formula (III) by determining the expression level of at least one biomarker selected from CD10, BCL6, MUM1 or any combination thereof; and administer to the individual a therapeutically effective amount of the compound of Formula (III) if there is an increase in expression level in at least one biomarker selected from CD10, BCL6, or a combination thereof and optionally a decrease in expression level of MUM1.

In some aspects, the subject is monitored every month, every 2 months, every 3 months, every 4 months, every 5 months, every 6 months, every 7 months, every 8 months, every 9 months, every 10 months, every 11 months, or every year to determine the level of expression of biomarker genes disclosed herein.

### Biomarkers Based on Gene Modifications

In some aspects, the methods disclosed herein is based on detecting the precense or absence of modification is a base substitution, an insertion, a deletion, a DNA rearrangement, a translocation, a copy number alteration, or a combination thereof.

The methods provided herein relate to detecting modifications in MYD88, CD79B, PIM1, CDKN2A, HLA-B, OSBPL10, ETV6, SPIB, TOX, BTG1, BTG2, HLA-A, SETD1B, HLA-C, MPEG1, FOXC1, TBL1XR1, KLHL14, GRHPR, CD58, PRDM1, VMP1, PIM2, WEE1, BCL11A, CHST2, ARID5B, HASPIN, IL16, PPP1R9B, or HNF1B as a predictive biomarker for identifying responder populations, especially those patients that are likely to be sensitive to treatment with the compound of Formula (III). The methods provided herein provide clinical advantages to the diagnosis and treatment of B-cell lymphomas, including easy access to samples, given that the modifications to biomarker genes described herein can reliably detected.

In some aspects, a method of treating DLBCL in a subject comprises: (a) determining the presence or absence of a modification in one or more biomarker genes in a subject, the biomarker genes selected from MYD88, CD79B, PIM1, CDKN2A, HLA-B, OSBPL10, ETV6, SPIB, TOX, BTG1, BTG2, HLA-A, SETD1B, HLA-C, MPEG1, FOXC1, TBL1XR1, KLHL14, GRHPR, CD58, PRDM1, VMP1, PIM2, WEE1, BCL11A, CHST2, ARID5B, HASPIN, IL16, PPP1R9B, or HNF1B; and (b) administering to the subject a therapeutically effective amount of compound of Formula (III) if there is a presence of a modification in the one or more biomarker genes. In some embodiments, the DLBCL subtype is activated B-cell diffuse large B-cell lymphoma (ABC-DLBCL), germinal center B-cell diffuse large B-cell lymphoma (GCB-DLBCL) or non-germinal center B-cell diffuse large B-cell lymphoma (non-GCB-DLBCL).

In some aspects, a method of monitoring whether a subject receiving the compound of Formula (III) for treatment of DLBCL has developed or is likely to develop resistance to the therapy comprises: (a) determining the presence or absence of a modification in one or more biomarker genes in a subject, the biomarker genes selected from MYD88, CD79B, PIM1, CDKN2A, HLA-B, OSBPL10, ETV6, SPIB, TOX, BTG1, BTG2, HLA-A, SETD1B, HLA-C, MPEG1, FOXC1, TBL1XR1, KLHL14, GRHPR, CD58, PRDM1, VMP1, PIM2, WEE1, BCL11A, CHST2, ARID5B, HASPIN, IL16, PPP1R9B, or HNF1B, wherein the subject is likely to develop resistance to the therapy if there is a presence of a modification in the one or more biomarker genes. In some embodiments, the DLBCL subtype is activated B-cell diffuse large B-cell lymphoma (ABC-DLBCL), germinal center B-cell diffuse large B-cell lymphoma (GCB-DLBCL) or non-germinal center B-cell diffuse large B-cell lymphoma (non-GCB-DLBCL).

In some aspects, a method of predicting a response in a subject receiving the compound of Formula (III) for treatment of DLBCL comprises: determining the presence or absence of a modification in one or more biomarker genes in a subject, the biomarker genes selected from MYD88, CD79B, PIM1, CDKN2A, HLA-B, OSBPL10, ETV6, SPIB, TOX, BTG1, BTG2, HLA-A, SETD1B, HLA-C, MPEG1, FOXC1, TBL1XR1, KLHL14, GRHPR, CD58, PRDM1, VMP1, PIM2, WEE1, BCL11A, CHST2, ARID5B, HASPIN, IL16, PPP1R9B, or HNF1B, wherein the subject is likely to respond to the therapy if there is a presence of a modification in the one or more biomarker genes. In some embodiments, the DLBCL subtype is activated B-cell diffuse large B-cell lymphoma (ABC-DLBCL), germinal center B-cell diffuse large B-cell lymphoma (GCB-DLBCL) or non-germinal center B-cell diffuse large B-cell lymphoma (non-GCB-DLBCL).

Further disclosed herein, in some aspects, is a method of optimizing the therapy of a subject receiving the compound of Formula (III) for treatment of diffuse large B cell lymphoma (DLBCL), comprising: (a) determining the presence or absence of a modification in one or more biomarker genes in a subject, the biomarker genes selected from MYD88, CD79B, PIM1, CDKN2A, HLA-B, OSBPL10, ETV6, SPIB, TOX, BTG1, BTG2, HLA-A, SETD1B, HLA-C, MPEG1, FOXC1, TBL1XR1, KLHL14, GRHPR, CD58, PRDM1, VMP1, PIM2, WEE1, BCL11A, CHST2, ARID5B, HASPIN, IL16, PPP1R9B, or HNF1B; (b) modifying the treatment based on the presence or absence of a modification in the one or more biomarker genes. In some embodiments, the DLBCL subtype is activated B-cell diffuse large B-cell lymphoma (ABC-DLBCL), germinal center B-cell diffuse large B-cell lymphoma (GCB-DLBCL) or non-germinal center B-cell diffuse large B-cell lymphoma (non-GCB-DLBCL).

In some aspects, a method of evaluating a treatment with the compound of Formula (III) in a subject comprises (a) determining the presence or absence of a modification in one or more biomarker genes in a subject, the biomarker genes selected from MYD88, CD79B, PIM1, CDKN2A, HLA-B, OSBPL10, ETV6, SPIB, TOX, BTG1, BTG2, HLA-A, SETD1B, HLA-C, MPEG1, FOXC1, TBL1XR1, KLHL14, GRHPR, CD58, PRDM1, VMP1, PIM2, WEE1, BCL11A, CHST2, ARID5B, HASPIN, IL16, PPP1R9B, or HNF1B; (b) modifying, discontinuing, or continuing the treatment based on the presence or absence of a modification in the one or more biomarker genes. In some embodiments, the treatment regimen is modified. In some embodiments, the dosage of the compound of Formula (III) is increased. In some embodiments, the dosage of the compound of Formula (III) is decreased. In some embodiments, the dosage of the compound of Formula (III) is not modified. In some embodiments, the frequency of administration of the compound of Formula (III) is increased. In some embodiments, the frequency of administration of the compound of Formula (III) is decreased. In some embodiments, the frequency of administration of the compound of Formula (III) is not modified. In some embodiments, the administration of the compound of Formula (III) is discontinued.

Also disclosed herein are methods for selecting a subject having diffuse large B cell lymphoma (DLBCL) for treatment with the compound of Formula (III) comprising: (a) determining the presence or absence of a modification in one or more biomarker genes in a subject, the biomarker genes selected from MYD88, CD79B, PIM1, CDKN2A, HLA-B, OSBPL10, ETV6, SPIB, TOX, BTG1, BTG2, HLA-A, SETD1B, HLA-C, MPEG1, FOXC1, TBL1XR1, KLHL14, GRHPR, CD58, PRDM1, VMP1, PIM2, WEE1, BCL11A, CHST2, ARID5B, HASPIN, IL16, PPP1R9B, or HNF1B; (b) selecting the subject if there is a presence of a modification in the one or more biomarker genes and administering to the subject a therapeutically effective amount of compound of Formula (III). In some embodiments, the DLBCL subtype is activated B-cell diffuse large B-cell lymphoma (ABC-DLBCL), germinal center B-cell diffuse large B-cell lymphoma (GCB-DLBCL) or non-germinal center B-cell diffuse large B-cell lymphoma (non-GCB-DLBCL).

In some aspects, a subject having DLBCL is characterized as resistant or is likely to become resistant to therapy with the compound of Formula (III) if the subject has the modification in one or more biomarker genes selected from MYD88, CD79B, PIM1, CDKN2A, HLA-B, OSBPL10, ETV6, SPIB, TOX, BTG1, BTG2, HLA-A, SETD1B, HLA-C, MPEG1, FOXC1, TBL1XR1, KLHL14, GRHPR, CD58, PRDM1, VMP1, PIM2, WEE1, BCL11A, CHST2, ARID5B, HASPIN, IL16, PPP1R9B, or HNF1B. In some embodiments, the DLBCL subtype is activated B-cell diffuse large B-cell lymphoma (ABC-DLBCL), germinal center B-cell diffuse large B-cell lymphoma (GCB-DLBCL) or non-germinal center B-cell diffuse large B-cell lymphoma (non-GCB-DLBCL).

In some aspects, the activated B-cell diffuse large B-cell lymphoma subtype (ABC-DLBCL) of DLBCL is characterized by one or more modifications to MYD88, CD79B, PIM1, CDKN2A, HLA-B, OSBPL10, ETV6, SPIB, TOX, BTG1, BTG2, HLA-A, SETD1B, HLA-C, MPEG1, FOXC1, TBL1XR1, KLHL14, GRHPR, CD58, PRDM1, VMP1, PIM2, WEE1, BCL11A, CHST2, ARID5B, HASPIN, IL16, PPP1R9B, or HNF1B. The modifications may be base substitution, insertion, deletion, DNA rearrangement, translocation, copy number alteration, or a combination thereof.

In some aspects, the germinal center (GCB) subtype of DLBCL is characterized by one or more modifications to MYD88, CD79B, PIM1, CDKN2A, HLA-B, OSBPL10, ETV6, SPIB, TOX, BTG1, BTG2, HLA-A, SETD1B, HLA-C, MPEG1, FOXC1, TBL1XR1, KLHL14, GRHPR, CD58, PRDM1, VMP1, PIM2, WEE1, BCL11A, CHST2, ARID5B, HASPIN, IL16, PPP1R9B, or HNF1B. The modifications may be base substitution, insertion, deletion, DNA rearrangement, translocation, copy number alteration, or a combination thereof.

In some aspects, the non-germinal center (non-GCB) subtype of DLBCL is characterized by one or more modifications to MYD88, CD79B, PIM1, CDKN2A, HLA-B, OSBPL10, ETV6, SPIB, TOX, BTG1, BTG2, HLA-A, SETD1B, HLA-C, MPEG1, FOXC1, TBL1XR1, KLHL14, GRHPR, CD58, PRDM1, VMP1, PIM2, WEE1, BCL11A, CHST2, ARID5B, HASPIN, IL16, PPP1R9B, or HNF1B. The modifications may be base substitution, insertion, deletion, DNA rearrangement, translocation, copy number alteration, or a combination thereof.

In some aspects, the sample for use in the methods is from any tissue or fluid from a patient. Samples include, but are not limited, to whole blood, dissociated bone marrow, bone marrow aspirate, pleural fluid, peritoneal fluid, central spinal fluid, abdominal fluid, pancreatic fluid, cerebrospinal fluid, brain fluid, ascites, pericardial fluid, urine, saliva, bronchial lavage, sweat, tears, ear flow, sputum, hydrocele fluid, semen, vaginal flow, milk, amniotic fluid, and secretions of respiratory, intestinal or genitourinary tract. In particular aspects, the sample is a blood serum sample. In particular aspects, the sample is a tumor biopsy sample. In particular aspects, the sample is from a fluid or tissue that is part of, or associated with, the lymphatic system or circulatory system. In some aspects, the sample is a blood sample that is a venous, arterial, peripheral, tissue, cord blood sample. In particular aspects, the sample is a blood cell sample containing one or more peripheral blood mononuclear cells (PBMCs). In some aspects, the sample contains one or more circulating tumor cells (CTCs). In some aspects, the sample contains one or more disseminated tumor cells (DTC, e.g. in a bone marrow aspirate sample).

In some aspects, the samples are obtained from the subject by any suitable means of obtaining the sample using well-known and routine clinical methods. Procedures for obtaining fluid samples from a subject are well known. For example, procedures for drawing and processing whole blood and lymph are well-known and can be employed to obtain a sample for use in the methods provided. Typically, for collection of a blood sample, an anti-coagulation agent (e.g. EDTA, or citrate and heparin or CPD (citrate, phosphate, dextrose) or comparable substances) is added to the sample to prevent coagulation of the blood. In some examples, the blood sample is collected in a collection tube that contains an amount of EDTA to prevent coagulation of the blood sample.

In some aspects, the collection of a sample from the subject is performed at regular intervals, such as, for example, one day, two days, three days, four days, five days, six days, one week, two weeks, weeks, four weeks, one month, two months, three months, four months, five months, six months, one year, daily, weekly, bimonthly, quarterly, biyearly or yearly. In some aspects, a sample is collected at a predetermined time or at regular intervals prior to, during, or following treatment or between successive treatments with the anti-cancer agent. In particular examples, a sample is obtained from the subject prior to administration of an anti-cancer therapy and then again at regular intervals after treatment has been effected.

In some aspects, the collection of a sample is performed at a predetermined time or at regular intervals relative to treatment with a compound of Formula (III). For example, a sample is collected from a patient at a predetermined time or at regular intervals prior to, during, or following treatment or between successive treatments with the compound of Formula (III). In particular examples, a sample is obtained from a patient prior to administration of a compound of Formula (III) and then again at regular intervals after treatment with the compound of Formula (III) has been effected. In some aspects, the patient is administered a compound of Formula (III) and one or more additional anti-cancer agents.

In some aspects, the sample is obtained at 1 week, 2 weeks, 3 weeks, 1 month, 2 months, 3 months, 4 months, 5 months, 6 months, 7 months, 8 months, 9 months, 10 months, 11 months, 12 months, 14 months, 16 months, 18 months, 20 months, 22 months, 24 months, 26 months, 28 months, 30 months, 32 months, 34 months, 36 months or longer following the first administration of the compound of Formula (III). In some aspects, the sample is obtained at 1 week, 2 weeks, 3 weeks, 1 month, 2 months, 3 months, 4 months, 5 months, 6 months, 7 months, 8 months, 9 months, 10 months, 11 months, 12 months, 14 months, 16 months, 18 months, 20 months, 22 months, 24 months, 26 months, 28 months, 30 months, 32 months, 34 months, 36 months or longer following the first administration of a compound of Formula (III) to a subject having DLBCL or ABC-DLBCL. In some aspects, the sample is obtained 1, 2, 3, 4, 5, 6, 7, 8, 9, 10 times or more over the course of treatment with a compound of Formula (III). In some aspects, the subject is responsive the treatment with a compound of Formula (III) when it is first administered.

In some aspects, the sample is obtained at 1 week, 2 weeks, 3 weeks, 1 month, 2 months, 3 months, 4 months, 5 months, 6 months, 7 months, 8 months, 9 months, 10 months, 11 months, 12 months, 14 months, 16 months, 18 months, 20 months, 22 months, 24 months, 26 months, 28 months, 30 months, 32 months, 34 months, 36 months or longer following the first administration of the compound of Formula (III).

In preferred aspects, the compound of Formula (III) can be used to treat DLBCL. In yet other aspects, compounds of the disclosure can be used to treat ABC-DLBCL, GCB-DLBCL or non-GCB-DLBCL.

In treatment methods according to the disclosure, an effective amount of a pharmaceutical agent according to the disclosure is administered to a subject suffering from or diagnosed as having such a disease, disorder, or condition. A “therapeutically effective amount” means an amount or dose sufficient to generally bring about the desired therapeutic benefit in patients in need of such treatment for the designated disease, disorder, or condition. Therapeutically effective amounts or doses of the compounds of the present disclosure may be ascertained by routine methods such as modeling, dose escalation studies or clinical trials, and by taking into consideration routine factors, e.g., the mode or route of administration or drug delivery, the pharmacokinetics of the compound, the severity and course of the disease, disorder, or condition, the subject's previous or ongoing therapy, the subject's health status and response to drugs, and the judgment of the treating physician. An example of a dose is in the range of from about 0.0001 to about 1,000 mg of compound per kg of subject's body weight per day, preferably about 0.05 to 100 mg/kg/day, or about 1 to 35 mg/kg/day, in single or divided dosage units (e.g., BID, TID, QID). For a 70-kg human, an illustrative range for a suitable dosage amount is from about 0.05 to about 7 g/day, or about 0.2 to about 2.5 g/day.

In some aspects, the therapeutically effective amount of the compound of Formula (III) is from about 0.0001 mg to about 10,000 mg. In some aspects, the therapeutically effective amount of the compound of Formula (III) is from about 0.0001 mg to about 1,000 mg.

In some aspects, the therapeutically effective amount of the compound of Formula (III) is from about 0.0001 mg to about 100 mg. In some aspects, the therapeutically effective amount of the compound of Formula (III) is from about 100 mg to about 200 mg. In some aspects, the therapeutically effective amount of the compound of Formula (III) is from about 100 mg to about 300 mg. In some aspects, the therapeutically effective amount of the compound of Formula (III) is from about 100 mg to about 400 mg. In some aspects, the therapeutically effective amount of the compound of Formula (III) is from about 100 mg to about 500 mg. In some aspects, the therapeutically effective amount of the compound of Formula (III) is from about 100 mg to about 600 mg. In some aspects, the therapeutically effective amount of the compound of Formula (III) is from about 100 mg to about 700 mg. In some aspects, the therapeutically effective amount of the compound of Formula (III) is from about 100 mg to about 800 mg. In some aspects, the therapeutically effective amount of the compound of Formula (III) is from about 100 mg to about 900 mg. In some aspects, the therapeutically effective amount of the compound of Formula (III) is from about 100 mg to about 1,000 mg. In some aspects, the therapeutically effective amount of the compound of Formula (III) is from about 140 mg to about 560 mg. In some aspects, the therapeutically effective amount of the compound of Formula (III) is about 140 mg. In some aspects, the therapeutically effective amount of the compound of Formula (III) is about 280 mg. In some aspects, the therapeutically effective amount of the compound of Formula (III) is about 560 mg.

In some aspects, the therapeutically effective amount of the compound of Formula (III) is administered once a day. In some aspects, the therapeutically effective amount of the compound of Formula (III) is administered twice a day. In some aspects, the therapeutically effective amount of the compound of Formula (III) is administered three times a day. In some aspects the therapeutically effective amount of the compound of formula (III) is administered orally.

In some aspects, maintenance therapy comprises administration of a daily dosage of a compound of Formula (III). In some aspects, maintenance therapy comprises multiple cycles of administration of a compound of Formula (III). In some aspects, a cycle of administration is one month, 2 months, 3 months, 4 months, 6 months, 6 months, 7 months, 8 months, 9 months, 10 months, 11 months, 12 months or longer. In some aspects, a cycle of administration comprises administration of a single therapeutic dosage of a compound of Formula (III) over the cycle. In some aspects, a cycle of administration comprises two or more different dosages of a compound of Formula (III) over the cycle. In some aspects, the dosage of a compound of Formula (III) differs over consecutive cycles. In some aspects, the dosage of a compound of Formula (III) increases over consecutive cycles. In some aspects, the dosage of a compound of Formula (III) is the same over consecutive cycles.

In addition, the compounds of the disclosure may be used in combination with additional active ingredients in the treatment of the above conditions. The additional active ingredients may be coadministered separately with a compound of the disclosure or included with such an agent in a pharmaceutical composition according to the disclosure. The combination may serve to increase efficacy (e.g., by including in the combination a compound potentiating the potency or effectiveness of an active agent according to the disclosure), decrease one or more side effects, or decrease the required dose of the active agent according to the disclosure.

The compounds of the disclosure are used, alone or in combination with one or more additional active ingredients, to formulate pharmaceutical compositions of the disclosure. A pharmaceutical composition of the disclosure comprises: (a) an effective amount of at least one compound in accordance with the disclosure; and (b) a pharmaceutically acceptable excipient.

In some aspects, the compound of Formula (III) can be administered in combination with one or more additional therapeutic agent. In some aspects, the one of more additional therapeutic agents are cyclophosphamide, doxorubicin, vincristine, prednisone and rituximab (R-CHOP).

In some aspects, where the subject is suffering from DLBCL, or any subset thereof, an anti-cancer agent is administered to the subject in addition to the compound of Formula (III). In one aspect, the anti-cancer agent is an inhibitor of mitogen-activated protein kinase signaling, e.g., U0126, PD98059, PD184352, PD0325901, ARRY-142886, SB239063, SP600125, BAY 43-9006, wortmannin, or LY294002. When used for the treatment of cancer, the compound of Formula (III) can be administered as a single agent. Alternatively, when used for the treatment of cancer or a malignancy, the compounds of Formula (III) can be administered in combination with other agents known to be useful for the treatment of cancer.

In some aspects, the compound of Formula (III) can be administered in combination with one or more additional therapeutic agent. In some aspects, the one of more additional therapeutic agent is a Bcl2 inhibitor. In some aspects, the Bcl2 inhibitor is 4-(4-{[2-(4-chlorophenyl)-4,4-dimethylcyclohex-1-en-1-yl]methyl}piperazin-1-yl)-N-({3-nitro-4-[(tetrahydro-2H-pyran-4-ylmethyl)amino]phenyl}sulfonyl)-2-(1H-pyrrolo[2,3-b]pyridin5-yloxy)benzamide) also known as venetoclax. In some aspects, 4-(4-{[2-(4-chlorophenyl)-4,4-dimethylcyclohex-1-en-1-yl]methyl}piperazin-1-yl)-N-({3-nitro-4-[(tetrahydro-2H-pyran-4-ylmethyl)amino]phenyl}sulfonyl)-2-(1H-pyrrolo[2,3-b]pyridin5-yloxy)benzamide) is administerd according to a weekly ramp up dosage regimen comprising administering about 20mg/day for the first week, about 50 mg/day for the second week, about100 mg/day for the third week, 200 mg/day for the third week, and 400 mg/day for the fourth week and beyond. In some aspects, the 4-(4-{[2-(4-chlorophenyl)-4,4-dimethylcyclohex-1-en-1-yl]methyl}piperazin-1-yl)-N-({3-nitro-4-[(tetrahydro-2H-pyran-4-ylmethyl)amino]phenyl}sulfonyl)-2-(1H-pyrrolo[2,3-b]pyridin5-yloxy)benzamide) is administered orally. In some aspects, the compound of Formula (III) can be administered in combination with 4-(4-{[2-(4-chlorophenyl)-4,4-dimethylcyclohex-1-en-1-yl]methyl}piperazin-1-yl)-N-({3-nitro-4-[(tetrahydro-2H-pyran-4-ylmethyl)amino]phenyl}sulfonyl)-2-(1H-pyrrolo[2,3-b]pyridin5-yloxy)benzamide) and rituximab. In some aspects, the compound of Formula (III) can be administered in combination with 4-(4-{[2-(4-chlorophenyl)-4,4-dimethylcyclohex-1-en-1-yl]methyl}piperazin-1-yl)-N-({3-nitro-4-[(tetrahydro-2H-pyran-4-ylmethyl)amino]phenyl}sulfonyl)-2-(1H-pyrrolo [2,3-b]pyridin5-yloxy)benzamide) and obinutuzumab.

In some aspects, the compound of Formula (III) can be administered in combination with one or more additional therapeutic agents selected from the group consisting of a chemotherapeutic agent, a steroid, an immunotherapeutic agent, a targeted therapy, and any combination thereof. In some aspects, the one or more additional therapeutic agent includes, but is not limited to a B cell receptor pathway inhibitor B cell receptor signaling inhibitor, a PI3K inhibitor, an IAP inhibitor, an mTOR inhibitor, a radioimmunotherapeutic, a DNA damaging agent, a proteosome inhibitor, a histone deacetylase inhibitor, a protein kinase inhibitor, a hedgehog inhibitor, an Hsp90 inhibitor, a telomerase inhibitor, a Jakl/2 inhibitor, a protease inhibitor, a PKC inhibitor, a PARP inhibitor, and any combination thereof. In some aspects, the B cell receptor pathway inhibitor includes, but is not limited to a CD79A inhibitor, a CD79B inhibitor, a CD 19 inhibitor, a Lyn inhibitor, a Syk inhibitor, a PI3K inhibitor, a Blnk inhibitor, a PLCy inhibitor, a PKCP inhibitor, or a combination thereof. In some aspects, the one or more additional therapeutic agents include, but are not limited to chlorambucil, ifosphamide, doxorubicin, mesalazine, thalidomide, lenalidomide, temsirolimus, everolimus, fludarabine, fostamatinib, paclitaxel, docetaxel, ofatumumab, rituximab, dexamethasone, prednisone, CAL-101, ibritumomab, tositumomab, bortezomib, pentostatin, endostatin, cyclophosphamide, hydroxydaunorubicin, vincristine, prednisone, rituximab, bendamustine, etoposide, prednisolone, and any combination thereof. In some aspects, the one or more therapeutic agents is a nitrogen mustard including but not limited to bendamustine, chlorambucil, chlormethine, cyclophosphamide, ifosfamide, melphalan, prednimustine, trofosfamide; Alkyl Sulfonates such as, but not limited to busulfan, mannosulfan, treosulfan; Ethylene Imines, carboquone, thiotepa, triaziquone; Nitrosoureas such as, but not limited to carmustine, fotemustine, lomustine, nimustine, ranimustine, semustine, streptozocin; Epoxides such as, but not limited to etoglucid; Other Alkylating Agents such as, but not limited to dacarbazine, mitobronitol, pipobroman, temozolomide; Folic Acid Analogues such as, but not limited to methotrexate, permetrexed, pralatrexate, raltitrexed; Purine Analogs such as, but not limited to cladribine, clofarabine, fludarabine, mercaptopurine, nelarabine, tioguanine; Pyrimidine Analogs such as, but not limited to azacitidine, capecitabine, carmofur, cytarabine, decitabine, fluorouracil, gemcitabine, tegafur; Vinca; Alkaloids such as for example vinblastine, vincristine, vindesine, vinflunine, vinorelbine; Podophyllotoxin Derivatives such as, but not limited to etoposide, teniposide; Colchicine derivatives such as, but not limited to demecolcine; Taxanes such as, but not limited to docetaxel, paclitaxel, paclitaxel poliglumex; Other Plant Alkaloids and Natural Products such as, but not limited to trabectedin; Actinomycines such as, but not limited to dactinomycin; Antracyclines such as for example aclarubicin, daunorubicin, doxorubicin, epirubicin, idarubicin, mitoxantrone, pirarubicin, valrubicin, zorubincin; Other Cytotoxic Antibiotics such as, but not limited to bleomycin, ixabepilone, mitomycin, plicamycin; Platinum Compounds such as for example carboplatin, cisplatin, oxaliplatin, satraplatin; Methylhydrazines such as, but not limited to procarbazine; Sensitizers such as, but not limited to aminolevulinic acid, efaproxiral, methyl aminolevulinate, porfimer sodium, temoporfm; Protein Kinase Inhibitors such as, but not limited to dasatinib, erlotinib, everolimus, gefitinib, imatinib, lapatinib, nilotinib, pazonanib, sorafenib, sunitinib, temsirolimus; Other Antineoplastic Agents such as, but not limited to alitretinoin, altretamine, amzacrine, anagrelide, arsenic trioxide, asparaginase, bexarotene, bortezomib, celecoxib, denileukin diftitox, estramustine, hydroxycarbamide, irinotecan, lonidamine, masoprocol, miltefosein, mitoguazone, mitotane, oblimersen, pegaspargase, pentostatin, romidepsin, sitimagene ceradenovec, tiazofurine, topotecan, tretinoin, vorinostat; Estrogens such as, but not limited to diethylstilbenol, ethinylestradiol, fosfestrol, polyestradiol phosphate; Progestogens such as, but not limited to gestonorone, medroxyprogesterone, megestrol; Gonadotropin Releasing Hormone Analogs such as, but not limited to buserelin, goserelin, leuprorelin, triptorelin; Anti-Estrogens such as, but not limited to fulvestrant, tamoxifen, toremifene; Anti-Androgens such as, but not limited to bicalutamide, flutamide, nilutamide, Enzyme Inhibitors, aminoglutethimide, anastrozole, exemestane, formestane, letrozole, vorozole; Other Hormone Antagonists such as, but not limited to abarelix, degarelix; Immunostimulants such as, but not limited to histamine dihydrochloride, mifamurtide, pidotimod, plerixafor, roquinimex, thymopentin; Immunosuppressants such as, but not limited to everolimus, gusperimus, leflunomide, mycophenolic acid, sirolimus; Calcineurin Inhibitors such as, but not limited to ciclosporin, tacrolimus; Other Immunosuppressants such as, but not limited to azathioprine, lenalidomide, methotrexate, thalidomide; and Radiopharmaceuticals such as, but not limited to, iobenguane, interferons, interleukins, Tumor Necrosis Factors, Growth Factors, or the like., immunostimulants such as, but not limited to ancestim, filgrastim, lenograstim, molgramostim, pegfilgrastim, sargramostim; Interferons such as, but not limited to interferon alfa natural, interferon alfa-2a, interferon alfa-2b, interferon alfacon-1, interferon alfa-n1, interferon beta natural, interferon beta-1a, interferon beta-1b, interferon gamma, peginterferon alfa-2a, peginterferon alfa-2b; Interleukins such as, but not limited to aldesleukin, oprelvekin; Other Immunostimulants such as, but not limited to BCG vaccine, glatiramer acetate, histamine dihydrochloride, immunocyanin, lentinan, melanoma vaccine, mifamurtide, pegademase, pidotimod, plerixafor, poly I:C, poly ICLC, roquinimex, tasonermin, thymopentin; Immunosuppressants such as, but not limited to abatacept, abetimus, alefacept, antilymphocyte immunoglobulin (horse), antithymocyte immunoglobulin (rabbit), eculizumab, efalizumab, everolimus, gusperimus, leflunomide, muromab-CD3, mycophenolic acid, natalizumab, sirolimus; TNF alpha Inhibitors such as for example adalimumab, afelimomab, certolizumab pegol, etanercept, golimumab, infliximab; Interleukin Inhibitors such as, but not limited to anakinra, basiliximab, canakinumab, daclizumab, mepolizumab, rilonacept, tocilizumab, ustekinumab; Calcineurin Inhibitors such as, but not limited to ciclosporin, tacrolimus; Other Immunosuppressants such as, but not limited to azathioprine, lenalidomide, methotrexate, thalidomide, Adalimumab, Alemtuzumab, Basiliximab, Bevacizumab, Cetuximab, Certolizumab pegol, Daclizumab, Eculizumab, Efalizumab, Gemtuzumab, Ibritumomab tiuxetan, Infliximab, Muromonab-CD3, Natalizumab, Panitumumab, Ranibizumab, Rituximab, Tositumomab, Trastuzumab, Additional cancer treatment regimens include Monoclonal Antibodies such as, but not limited to alemtuzumab, bevacizumab, catumaxomab, cetuximab, edrecolomab, gemtuzumab, ofatumumab, panitumumab, rituximab, trastuzumab, Immunosuppressants, eculizumab, efalizumab, muromab-CD3, natalizumab; TNF alpha Inhibitors such as, but not limited to adalimumab, afelimomab, certolizumab pegol, golimumab, infliximab, Interleukin Inhibitors, basiliximab, canakinumab, daclizumab, mepolizumab, tocilizumab, ustekinumab, Radiopharmaceuticals, ibritumomab tiuxetan, tositumomab; Others Monoclonal Antibodies such as, but not limited to abagovomab, adecatumumab, alemtuzumab, anti-CD30 monoclonal antibody Xmab2513, anti-MET monoclonal antibody MetMab, apolizumab, apomab, arcitumomab, basiliximab, bispecific antibody 2B1, blinatumomab, brentuximab vedotin, capromab pendetide, cixutumumab, claudiximab, conatumumab, dacetuzumab, denosumab, eculizumab, epratuzumab, epratuzumab, ertumaxomab, etaracizumab, figitumumab, fresolimumab, galiximab, ganitumab, gemtuzumab ozogamicin, glembatumumab, ibritumomab, inotuzumab ozogamicin, ipilimumab, lexatumumab, lintuzumab, lintuzumab, lucatumumab, mapatumumab, matuzumab, milatuzumab, monoclonal antibody CC49, necitumumab, nimotuzumab, ofatumumab, oregovomab, pertuzumab, ramacurimab, ranibizumab, siplizumab, sonepcizumab, tanezumab, tositumomab, trastuzumab, tremelimumab, tucotuzumab celmoleukin, veltuzumab, visilizumab, volociximab, zalutumumab. Additional cancer treatment regimens include agents that affect the tumor micro-enviroment such as, but not limited to cellular signaling network (e.g. phosphatidylinositol 3-kinase (PI3K) signaling pathway, signaling from the B-cell receptor and the IgE receptor). In some aspects, the one or more therapeutic agent is a PI3K signaling inhibitor or a syc kinase inhibitor. In one aspect, the syk inhibitor is R788. In another aspect is a PKCy inhibitor such as, but not limited to, enzastaurin. Examples of agents that affect the tumor micro-environment include but are not limited to PI3K signaling inhibitor, syc kinase inhibitor, Protein Kinase Inhibitors such as, but not limited to dasatinib, erlotinib, everolimus, gefitinib, imatinib, lapatinib, nilotinib, pazonanib, sorafenib, sunitinib, temsirolimus; Other Angiogenesis Inhibitors such as, but not limited to GT-111, JI-101, R1530; Other Kinase Inhibitors such as for example AC220, AC480, ACE-041, AMG 900, AP24534, Arry-614, AT7519, AT9283, AV-951, axitinib, AZD1 152, AZD7762, AZD8055, AZD8931, bafetinib, BAY 73-4506, BGJ398, BGT226, BI 811283, BI6727, BIBF 1120, BIBW 2992, BMS-690154, BMS-777607, BMS-863233, BSK-461364, CAL-101, CEP-11981, CYC116, DCC-2036, dinaciclib, dovitinib lactate, E7050, EMD 1214063, ENMD-2076, fostamatinib disodium, GSK2256098, GSK690693, INCB18424, INNO-406, JNJ-26483327, JX-594, KX2-391, linifanib, LY2603618, MGCD265, MK-0457, MK1496, MLN8054, MLN8237, MP470, NMS-1116354, NMS-1286937, ON 01919.Na, OSI-027, OSI-930, Btk inhibitor, PF-00562271, PF-02341066, PF-03814735, PF-04217903, PF-04554878, PF-04691502, PF-3758309, PHA-739358, PLC3397, progenipoietin, R547, R763, ramucirumab, regorafenib, R05185426, SAR103168, S3333333CH 727965, SGI-1176, SGX523, SNS-314, TAK-593, TAK-901, TKI258, TLN-232, TTP607, XL147, XL228, XL281R05126766, XL418, XL765, inhibitors of mitogen-activated protein kinase signaling such as, but not limited to U0126, PD98059, PD184352, PD0325901, ARRY-142886, SB239063, SP600125, BAY 43-9006, wortmannin, or LY294002; Syk inhibitors; mTOR inhibitors; and antibodies (e.g., rituxan), Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; iimofosine; interleukin II (including recombinant interleukin II, or rlL2), interferon alfa-2a; interferon alfa-2b; interferon alfa-n1; interferon alfa-n3; interferon beta-1 a; interferon gamma-1 b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safmgol; safmgol hydrochloride; semustine; simtrazene; sparfosate sodium; sparsomycin; spiro germanium hydrochloride; spiromustine; spiroplatin; streptonigrin; streptozocin; sulofenur; talisomycin; tecogalan sodium; tegafur; teloxantrone hydrochloride; temoporfm; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfm; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride; 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide;hypericin; ibandronic acid; idarubicin; idoxifene; idramantone; ilmofosine; ilomastat; imidazoacridones; imiquimod; immunostimulant peptides; insulin-such as for example growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin;lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin;loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone Bl; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfm; vinorelbine; vinxaltine; vitaxin; vorozole; zanoterone; zeniplatin; zilascorb; and zinostatin stimalamer. Yet other anticancer agents that can be employed in combination with the compound of Formula (III) include alkylating agents, antimetabolites, natural products, or hormones, such as, but not limited to nitrogen mustards (such as, but not limited to mechloroethamine, cyclophosphamide, chlorambucil, etc.), alkyl sulfonates (such as, but not limited to busulfan), nitrosoureas (such as, but not limited to carmustine, lomusitne, etc.), or triazenes (decarbazine, etc.). Examples of antimetabolites include but are not limited to folic acid analog (such as, but not limited to methotrexate), or pyrimidine analogs (such as, but not limited to Cytarabine), purine analogs (such as, but not limited to mercaptopurine, thioguanine, pentostatin). Examples of alkylating agents that can be employed in combination the compounds of Formula (III) include, but are not limited to, nitrogen mustards (such as, but not limited to mechloroethamine, cyclophosphamide, chlorambucil, meiphalan, etc.), ethylenimine and methylmelamines (such as, but not limited to hexamethlymelamine, thiotepa), alkyl sulfonates (such as, but not limited to busulfan), nitrosoureas (such as, but not limited to carmustine, lomusitne, semustine, streptozocin, etc.), or triazenes (decarbazine, etc.). Examples of antimetabolites include, but are not limited to folic acid analog (such as, but not limited to methotrexate), or pyrimidine analogs (such as, but not limited to fluorouracil, floxouridine, Cytarabine), purine analogs (such as, but not limited to mercaptopurine, thioguanine, pentostatin. Examples of anti-cancer agents which act by arresting cells in the G2-M phases due to stabilized microtubules and which can be used in combination with a compound of Formula (III) compound include without limitation the following marketed drugs and drugs in development: Erbulozole (also known as R-55104), Dolastatin 10 (also known as DLS-10 and NSC-376128), Mivobulin isethionate (also known as CI-980), Vincristine, NSC-639829, Discodermolide (also known as NVP-XX-A-296), ABT-751 (Abbott, also known as E-7010), Altorhyrtins (such as Altorhyrtin A and Altorhyrtin C), Spongistatins (such as Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (also known as LU-103793 and NSC-D-669356), Epothilones (such as Epothilone A, Epothilone B, Epothilone C (also known as desoxyepothilone A or dEpoA), Epothilone D (also referred to as KOS-862, dEpoB, and desoxyepothilone B), Epothilone E, Epothilone F, Epothilone B N-oxide, Epothilone A N-oxide, 16-aza-epothilone B, 21-aminoepothilone B (also known as BMS-310705), 21-hydroxyepothilone D (also known as Desoxyepothilone F and dEpoF), 26-fluoroepothilone), Auristatin PE (also known as NSC-654663), Soblidotin (also known as TZT-1027), LS-4559-P (Pharmacia, also known as LS-4577), LS-4578 (Pharmacia, also known as LS-477-P), LS-4477 (Pharmacia), LS-4559 (Pharmacia), RPR-112378 (Aventis), Vincristine sulfate, DZ-3358 (Daiichi), FR-182877 (Fujisawa, also known as WS-9885B), GS-164 (Takeda), GS-198 (Takeda), KAR-2 (Hungarian Academy of Sciences), BSF-223651 (BASF, also known as ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (also known as LY-355703), AC-7739 (Ajinomoto, also known as AVE-8063A and CS-39.HCl), AC-7700 (Ajinomoto, also known as AVE-8062, AVE-8062A, CS-39-L-Ser.HCl, and RPR-258062A), Vitilevuamide, Tubulysin A, Canadensol, Centaureidin (also known as NSC-106969), T-138067 (Tularik, also known as T-67, TL-138067 and TI-138067), COBRA-1 (Parker Hughes Institute, also known as DDE-261 and WHI-261), H10 (Kansas State University), H16 (Kansas State University), Oncocidin Al (also known as BTO-956 and DIME), DDE-313 (Parker Hughes Institute), Fijianolide B, Laulimalide, SPA-2 (Parker Hughes Institute), SPA-1 (Parker Hughes Institute, also known as SPIKET-P), 3-IAABU (Cytoskeleton/Mt. Sinai School of Medicine, also known as MF-569), Narcosine (also known as NSC-5366), Nascapine, D-24851 (Asta Medica), A-105972 (Abbott), Hemiasterlin, 3-B A ABU (Cytoskeleton/Mt. Sinai School of Medicine, also known as MF-191), TMPN (Arizona State University), Vanadocene acetylacetonate, T-138026 (Tularik), Monsatrol, Inanocine (also known as NSC-698666), 3-1AABE (Cytoskeleton/Mt. Sinai School of Medicine), A-204197 (Abbott), T-607 (Tuiarik, also known as T-900607), RPR-115781 (Aventis), Eleutherobins (such as Desmethyleleutherobm, Desaetyleleutherobin, lsoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (also known as NSCL-96F037), D-68838 (Asta Medica), D-68836 (Asta Medica), Myoseverin B, D-43411 (Zentaris, also known as D-81862), A-289099 (Abbott), A-318315 (Abbott), HTI-286 (also known as SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi).

Delivery forms of the pharmaceutical compositions containing one or more dosage units of the active agents may be prepared using suitable pharmaceutical excipients and compounding techniques known or that become available to those skilled in the art. The compositions may be administered in the inventive methods by a suitable route of delivery, e.g., oral, parenteral, rectal, topical, or ocular routes, or by inhalation.

The preparation may be in the form of tablets, capsules, sachets, dragees, powders, granules, lozenges, powders for reconstitution, liquid preparations, or suppositories. Preferably, the compositions are formulated for intravenous infusion, topical administration, or oral administration.

For oral administration, the compounds of the disclosure can be provided in the form of tablets or capsules, or as a solution, emulsion, or suspension. To prepare the oral compositions, the compounds may be formulated to yield a dosage of, e.g., from about 0.05 to about 100 mg/kg daily, or from about 0.05 to about 35 mg/kg daily, or from about 0.1 to about 10 mg/kg daily. For example, a total daily dosage of about 5 mg to 5 g daily may be accomplished by dosing once, twice, three, or four times per day.

Oral tablets may include a compound according to the disclosure mixed with pharmaceutically acceptable excipients such as inert diluents, disintegrating agents, binding agents, lubricating agents, sweetening agents, flavoring agents, coloring agents and preservative agents. Suitable inert fillers include sodium and calcium carbonate, sodium and calcium phosphate, lactose, starch, sugar, glucose, methyl cellulose, magnesium stearate, mannitol, sorbitol, and the like. Exemplary liquid oral excipients include ethanol, glycerol, water, and the like. Starch, polyvinyl-pyrrolidone (PVP), sodium starch glycolate, microcrystalline cellulose, and alginic acid are suitable disintegrating agents. Binding agents may include starch and gelatin. The lubricating agent, if present, may be magnesium stearate, stearic acid or talc. If desired, the tablets may be coated with a material such as glyceryl monostearate or glyceryl distearate to delay absorption in the gastrointestinal tract, or may be coated with an enteric coating.

Capsules for oral administration include hard and soft gelatin capsules. To prepare hard gelatin capsules, compounds of the disclosure may be mixed with a solid, semi-solid, or liquid diluent. Soft gelatin capsules may be prepared by mixing the compound of the disclosure with water, an oil such as peanut oil or olive oil, liquid paraffin, a mixture of mono and di-glycerides of short chain fatty acids, polyethylene glycol 400, or propylene glycol.

Liquids for oral administration may be in the form of suspensions, solutions, emulsions or syrups or may be lyophilized or presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

The active agents of this disclosure may also be administered by non-oral routes. For example, the compositions may be formulated for rectal administration as a suppository. For parenteral use, including intravenous, intramuscular, intraperitoneal, or subcutaneous routes, the compounds of the disclosure may be provided in sterile aqueous solutions or suspensions, buffered to an appropriate pH and isotonicity or in parenterally acceptable oil. Suitable aqueous vehicles include Ringer's solution and isotonic sodium chloride. Such forms will be presented in unit-dose form such as ampules or disposable injection devices, in multi-dose forms such as vials from which the appropriate dose may be withdrawn, or in a solid form or pre-concentrate that can be used to prepare an injectable formulation. Illustrative infusion doses may range from about 1 to 1000 μg/kg/minute of compound, admixed with a pharmaceutical carrier over a period ranging from several minutes to several days.

For topical administration, the compounds may be mixed with a pharmaceutical carrier at a concentration of about 0.1% to about 10% of drug to vehicle. Another mode of administering the compounds of the disclosure may utilize a patch formulation to affect transdermal delivery.

Compounds of the disclosure may alternatively be administered in methods of this disclosure by inhalation, via the nasal or oral routes, e.g., in a spray formulation also containing a suitable carrier.

Compounds of the disclosure can be prepared using the knowledge of one skilled in the art in combination with the present disclosure. For example, compounds of the disclosure can be prepared according to the schemes and examples disclosed in U.S. Pat. Nos. 10,717,745, 10,934,310 and PCT application publication WO2017100662, each of which is hereby incorporated in its entirety.

For use in the diagnostic and therapeutic applications described herein, kits and articles of manufacture are also described herein. Such kits can comprise a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. The containers are formed from any acceptable material including, e.g., glass or plastic. In some aspects, the kits provided herein are for use in determining the level of expression of biomarker genes or modifictions of biomarker genes. In some aspects, the kits provided herein are for use as a companion diagnostic with a compound of Formula (III). In some aspects the kits are employed for selecting patients for treatment with a compound of Formula (III), for identifying subjects as sensitive to a compound of Formula (III) of for evaluating treatment with the compound of Formula (III). In some aspects the kits are employed for selecting patients for treatment with a compound of Formula (III), for identifying subjects as resistant or likely to become resistant to a compound of Formula (III), for monitoring the development of resistance to a compound of Formula (III), or combinations thereof.

The kits provided herein contain one or more reagents for the detection of CCL3, CCL4, ACTG2, LOR, GAPT, CCND2, SELL, GEN1, HDAC9, CD10, BCL6, MUM1, or any combination thereof, expression. In some embodiments, the kits provided herein contain one or more reagents for the detection of modifications of MYD88, CD79B, PIM1, CDKN2A, HLA-B, OSBPL10, ETV6, SPIB, TOX, BTG1, BTG2, HLA-A, SETD1B, HLA-C, MPEG1, FOXC1, TBL1XR1, KLHL14, GRHPR, CD58, PRDM1, VMP1, PIM2, WEE1, BCL11A, CHST2, ARID5B, HASPIN, IL16, PPP1R9B, or HNF1B. Exemplary reagents include but are not limited to, antibodies, buffers, nucleic acids, microarrays, ELISA plates, substrates for enzymatic staining, chromagens or other materials, such as slides, containers, microtiter plates, and optionally, instructions for performing the methods. Those of skill in the art will recognize many other possible containers and plates and reagents that can be used for contacting the various materials.

### ASPECTS

The disclosure is also directed to the following aspects:

### Aspect 1

A compound of formula (III):

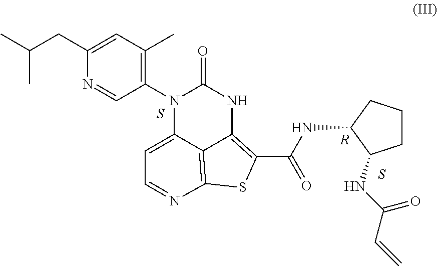

### Aspect 2

The compound of formula (III) of the preceding aspect that is a pharmaceutically acceptable salt, hydrate, polymorph or solvate thereof.

### Aspect 3

A pharmaceutical composition comprising a compound of formula (III), or a pharmaceutically acceptable salt, hydrate, polymorph or solvate thereof, and a pharmaceutically acceptable excipient.

### Aspect 4

A method of inhibiting Bruton's tyrosine kinase comprising contacting the kinase with a compound of formula (III).

### Aspect 5

A compound of Formula (III), or a pharmaceutically acceptable salt, hydrate, polymorph or solvate thereof for use in a method of treating DLBCL in a patient.

### Aspect 6

The use of aspect 5, wherein the DLBCL is ABC-DLBCL, germinal center B-cell diffuse large B-cell lymphoma (GCB-DLBCL) or non-germinal center B-cell diffuse large B-cell lymphoma (non-GCB-DLBCL).

### Aspect 7

A compound of Formula (III) for use in the treatment of DLBCL in a subject comprising (a) determining an expression level of CCL3, CCL4, ACTG2, LOR, GAPT, CCND2, SELL, GEN1, HDAC9, or any combination thereof, in a sample from the patient; and (b) administering a therapeutically effective amount compound of Formula (III) if the expression of CCL3, CCL4, ACTG2, LOR, GAPT, CCND2, SELL, GEN1, HDAC9, or any combination thereof, is increased relative to a control or reference level.

### Aspect 8

A compound of Formula (III) for use in the treatment of ABC-DLBCL in a subject comprising (a) determining an expression level of CCL3, CCL4, ACTG2, LOR, GAPT, CCND2, SELL, GEN1, HDAC9, or any combination thereof, in a sample from the patient; and (b) administering a therapeutically effective amount compound of Formula (III) if the expression of CCL3, CCL4, ACTG2, LOR, GAPT, CCND2, SELL, GEN1, HDAC9, or any combination thereof, is increased relative to a control or reference level.

### Aspect 9

The use of aspect 7, wherein the control or reference level is the level of expression of CCL3, CCL4, ACTG2, LOR, GAPT, CCND2, SELL, GEN1, HDAC9, or any combination thereof, in a normal patient.

### Aspect 10

A compound of Formula (III) for use in the treatment of GCB-DLBCL in a subject comprises (a) determining an expression level of CD10, BCL6, and MUM1, in a sample from the patient; and (b) administering a therapeutically effective amount of compound of Formula (III) if the expression of CD10 and BCL6, is increased relative to a control or reference level and expression of MUM1 is not increased relative to a control or reference level. In some aspects, a reference level is a level of expression of CD10, BCL6, and MUM1 in a normal patient.

### Aspect 11

A compound of Formula (III) for use in the treatment of DLBCL in a subject comprising:
(a) determining the presence or absence of a modification in one or more biomarker genes in a subject, the biomarker genes selected from MYD88, CD79B, PIM1, CDKN2A, HLA-B, OSBPL10, ETV6, SPIB, TOX, BTG1, BTG2, HLA-A, SETD1B, HLA-C, MPEG1, FOXC1, TBL1XR1, KLHL14, GRHPR, CD58, PRDM1, VMP1, PIM2, WEE1, BCL11A, CHST2, ARID5B, HASPIN, IL16, PPP1R9B, or HNF1B; and (b) administering to the subject a therapeutically effective amount of the compound of Formula (III) if there is a presence of a modification in the one or more biomarker genes.

### Aspect 12

A compound of Formula (III) for use in selecting a subject having diffuse large B cell lymphoma (DLBCL) for treatment with the compound of Formula (III) comprising: (a) determining the presence or absence of a modification in one or more biomarker genes in a subject, the biomarker genes selected from MYD88, CD79B, PIM1, CDKN2A, HLA-B, OSBPL10, ETV6, SPIB, TOX, BTG1, BTG2, HLA-A, SETD1B, HLA-C, MPEG1, FOXC1, TBL1XR1, KLHL14, GRHPR, CD58, PRDM1, VMP1, PIM2, WEE1, BCL11A, CHST2, ARID5B, HASPIN, IL16, PPP1R9B, or HNF1B; and (b) selecting the subject if there is a presence of a modification in the one or more biomarker genes and administering to the subject a therapeutically effective amount of the compound of Formula (III).

### Aspect 13

A compound of Formula (III) for use in monitoring whether a subject receiving the compound of Formula (III) for treatment of DLBCL has developed or is likely to develop resistance to the therapy, comprising: determining the presence or absence of a modification in one or more biomarker genes in a subject, the biomarker genes selected from MYD88, CD79B, PIM1, CDKN2A, HLA-B, OSBPL10, ETV6, SPIB, TOX, BTG1, BTG2, HLA-A, SETD1B, HLA-C, MPEG1, FOXC1, TBL1XR1, KLHL14, GRHPR, CD58, PRDM1, VMP1, PIM2, WEE1, BCL11A, CHST2, ARID5B, HASPIN, IL16, PPP1R9B, or HNF1B, wherein the subject is likely to develop resistance to the therapy if there is a presence of a modification in the one or more biomarker genes.

### Aspect 14

A compound of Formula (III) for use in optimizing the therapy of a subject receiving the compound of Formula (III) for treatment of diffuse large B cell lymphoma (DLBCL), comprising: (a) determining the presence or absence of a modification in one or more biomarker genes in a subject, the biomarker genes selected from CARD11, CD79A, CD79B, BCL10, KLHL6, BTK, SYK, NFKBIA, TNFAIP3, CDKN2A, CDKN2B, SMARCA4, TNFRSF14, HIST1H1D, ARID1A, EPHA3, ASTML, MYD88, MLL2, FOXO1, PCLO, TP53, ICK, MAP3K13, HIST1H1E, SOCS1, MTOR, TBL1XR1, BTG1, NOTCH2, SPEN, PLCG, NFKBIZ, NFKBID, ATM, BCL2, CXCR4, EZH2, KMT2D, NOTCH1, PLCG2, ZC3H12D, ZC3H12A, RC3H1, CYLD, N4BP1, RELB, and RBCK1; and (b) modifying the treatment based on the presence or absence of a modification in the one or more biomarker genes.

### Aspect 15

The use of aspects 10-14, wherein the modification in or more biomarker genes comprises a base substitution, an insertion, a deletion, a DNA rearrangement, a translocation, a copy number alteration, or a combination thereof.

### Aspect 16

The use of aspects 5 to 15, wherein the therapeutically effective amount of the compound of Formula (III) is about 140 mg to about 560 mg.

### Aspect 17

The use of aspects 5 to 15, wherein the therapeutically effective amount of the compound of Formula (III) is about 140 mg.

### Aspect 18

The use of aspects 5 to 15, wherein the therapeutically effective amount of the compound of Formula (III) is about 280 mg.

### Aspect 19

The use of aspects 5 to 15, wherein the therapeutically effective amount of the compound of Formula (III) is about 560 mg.

### Aspect 20

The use of aspects 5 to 19, wherein the therapeutically effective amount of the compound of Formula (III) is administered once a day.

### Aspect 21

The use of aspects 5 to 19, wherein the therapeutically effective amount of the compound of Formula (III) is administered twice a day.

### Aspect 22

The use of aspects 5 to 19, wherein the therapeutically effective amount of the compound of Formula (III) is administered three times a day.

### Aspect 23

The use of aspects 5 to 22, wherein the compound of formula (III) is administered orally.

### Aspect 24

The use of aspects 5 to 23, further comprising administering 4-(4-{[2-(4-chlorophenyl)-4,4-dimethylcyclohex-1-en-1-yl]methyl}piperazin-1-yl)-N-({3-nitro-4-[(tetrahydro-2H-pyran-4-ylmethy)amino]phenyl}sulfonyl)-2-(1H-pyrrolo[2,3-b] pyridin5-yloxy)benzamide).

### Aspect 25

The use of aspect 24, wherein the 4-(4-{[2-(4-chlorophenyl)-4,4-dimethylcyclohex-1-en-1-yl]methyl}piperazin-1-yl)-N-({3-nitro-4-[(tetrahydro-2H-pyran-4-ylmethyl)amino]phenyl}sulfonyl)-2-(1H-pyrrolo[2,3-b]pyridin5-yloxy)benzamide) is administerd according to a weekly ramp up dosage regimen comprising administering about 20 mg/day for the first week, about 50 mg/day for the second week, about100 mg/day for the third week, 200 mg/day for the third week, and 400 mg/day for the fourth week and beyond.

### Aspect 26

The use of aspects 5 to 23, further comprising administering cyclophosphamide, doxorubicin, vincristine, prednisone and rituximab.

What is claimed:

1. A method of treating DLBCL in a subject comprising (a) determining an expression level of CCL3, CCL4, ACTG2, LOR, GAPT, CCND2, SELL, GEN1, HDAC9, or any combination thereof, in a sample from the patient; and (b) administering a therapeutically effective amount compound of Formula (III): 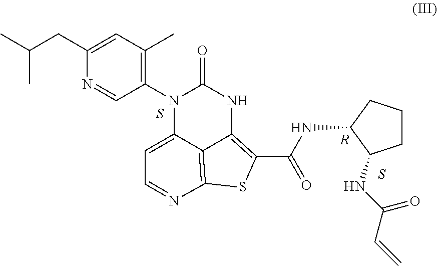
if the expression of CCL3, CCL4, ACTG2, LOR, GAPT, CCND2, SELL, GEN1, HDAC9, or any combination thereof, is increased relative to a control or reference level.

2. The method of claim 1, wherein the DLBCL is ABC-DLBCL.

3. The method of claim 1, wherein the control or reference level is the level of expression of CCL3, CCL4, ACTG2, LOR, GAPT, CCND2, SELL, GEN1, HDAC9, or any combination thereof, in a normal patient.

4. A method of treating GCB-DLBCL in a subject comprises (a) determining an expression level of CD10, BCL6, and MUM1, in a sample from the patient; and (b) administering a therapeutically effective amount of compound of Formula (III): 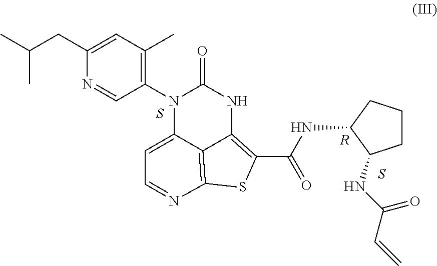
if the expression of CD10 and BCL6, is increased relative to a control or reference level and expression of MUM1 is not increased relative to a control or reference level.

5. The The method of claim 4, wherein the reference level is a level of expression of CD10, BCL6, and MUM1, in a normal patient.

6. A method of treating DLBCL in a subject comprising: (a) determining the presence or absence of a modification in one or more biomarker genes in a subject, the biomarker genes selected from MYD88, CD79B, PIM1, CDKN2A, HLA-B, OSBPL10, ETV6, SPIB, TOX, BTG1, BTG2, HLA-A, SETD1B, HLA-C, MPEG1, FOXC1, TBL1XR1, KLHL14, GRHPR, CD58, PRDM1, VMP1, PIM2, WEE1, BCL11A, CHST2, ARID5B, HASPIN, IL16, PPP1R9B, or HNF1B; and (b) administering to the subject a therapeutically effective amount of the compound of Formula (III) if there is a presence of a modification in the one or more biomarker genes.

7. A method for selecting a subject having diffuse large B cell lymphoma (DLBCL) for treatment with the compound of Formula (III) comprising: (a) determining the presence or absence of a modification in one or more biomarker genes in a subject, the biomarker genes selected from MYD88, CD79B, PIM1, CDKN2A, HLA-B, OSBPL10, ETV6, SPIB, TOX, BTG1, BTG2, HLA-A, SETD1B, HLA-C, MPEG1, FOXC1, TBL1XR1, KLHL14, GRHPR, CD58, PRDM1, VMP1, PIM2, WEE1, BCL11A, CHST2, ARID5B, HASPIN, IL16, PPP1R9B, or HNF1B; and (b) selecting the subject if there is a presence of a modification in the one or more biomarker genes and administering to the subject a therapeutically effective amount of the compound of Formula (III).

8. A method of monitoring whether a subject receiving the compound of Formula (III) for treatment of DLBCL has developed or is likely to develop resistance to the therapy, comprising:
determining the presence or absence of a modification in one or more biomarker genes in a subject, the biomarker genes selected from MYD88, CD79B, PIM1, CDKN2A, HLA-B, OSBPL10, ETV6, SPIB, TOX, BTG1, BTG2, HLA-A, SETD1B, HLA-C, MPEG1, FOXC1, TBL1XR1, KLHL14, GRHPR, CD58, PRDM1, VMP1, PIM2, WEE1, BCL11A, CHST2, ARID5B, HASPIN, IL16, PPP1R9B, or HNF1B, wherein the subject is likely to develop resistance to the therapy if there is a presence of a modification in the one or more biomarker genes.

9. A method of optimizing the therapy of a subject receiving the compound of Formula (III) for treatment of diffuse large B cell lymphoma (DLBCL), comprising: (a) determining the presence or absence of a modification in one or more biomarker genes in a subject, the biomarker genes selected from CARD11, CD79A, CD79B, BCL10, KLHL6, BTK, SYK, NFKBIA, TNFAIP3, CDKN2A, CDKN2B, SMARCA4, TNFRSF14, HIST1H1D, ARID1A, EPHA3, ASTML, MYD88, MLL2, FOXO1, PCLO, TP53, ICK, MAP3K13, HIST1H1E, SOCS1, MTOR, TBL1XR1, BTG1, NOTCH2, SPEN, PLCG, NFKBIZ, NFKBID, ATM, BCL2, CXCR4, EZH2, KMT2D, NOTCH1, PLCG2, ZC3H12D, ZC3H12A, RC3H1, CYLD, N4BP1, RELB, and RBCK1; and (b) modifying the treatment based on the presence or absence of a modification in the one or more biomarker genes.

10. The method of any one of claims 1-9, wherein the therapeutically effective amount of the compound of Formula (III) is from about 140 mg to about 560 mg.

11. The method of any one of claims 1-9, wherein the therapeutically effective amount of the compound of Formula (III)is about 140 mg.

12. The method of any one of claims 1-9, wherein the therapeutically effective amount of the compound of Formula (III) is about 280 mg.

13. The method of any one of claims 1-9, wherein the therapeutically effective amount of the compound of Formula (III) is about 560 mg.

14. The method of any one of claims 1-13, wherein the therapeutically effective amount of the compound of Formula (III) is administered once a day.

15. The method of any one of claims 1-13, wherein the therapeutically effective amount of the compound of Formula (III) is administered twice a day.

16. The method of any one of claims 1-13, wherein the therapeutically effective amount of the compound of Formula (III) is administered three times a day.

17. The method of any one of claims 1-16, wherein the compound of formula (III) is administered orally.

18. The method of any one of claims 1-9, further comprising administering 4-(4-{[2-(4-chlorophenyl)-4,4-dimethylcyclohex-1-en-1-yl]methyl}piperazin-1-yl)-N-({3-nitro-4-[(tetrahydro-2H-pyran-4-ylmethyl)amino]phenyl}sulfonyl)-2-(1H-pyrrolo[2,3-b]pyridin5-yloxy)benzamide).

19. The method of any one of claims 1-9, further comprising administering cyclophosphamide, doxorubicin, vincristine, prednisone and rituximab.
